(19) United States
(12) Patent Application Publication
(10) Pub. No.: US 2006/0009700 A1
(43) Pub. Date: Jan. 12, 2006

(54) Apparatus and method for assessing peripheral circulation to evaluate a physiological condition

(75) Inventors: Anne M. Brumfield, Morgantown, WV (US); Aaron W. Schopper, Morgantown, WV (US); Michael E. Andrew, Morgantown, WV (US)

Correspondence Address: KLARQUIST SPARKMAN, LLP, 121 S.W. SALMON STREET, SUITE 1600, PORTLAND, OR, 97204, US

(73) Assignee: The Gov. of the U.S.A as represented by the Secrety of the Dept. of H.H.S., Centers for D.C.P.

(21) Appl. No.: 11/148,962

(22) Filed: Jun. 8, 2005

Related U.S. Application Data
(60) Provisional application No. 60/578,174, filed on Jun. 8, 2004.

(51) Int. Cl. A61B 5/02; A61B 5/103

(52) U.S. Cl. 600/504; 600/587

(57) ABSTRACT

Peripheral blood flow signals of a feature can be acquired and analyzed to determine blood flow characteristics for evaluating a physiological condition. Paradigmatic characteristics of blood flow of the feature can be used to associate a physiological condition with a subject. Upon determination that a blood flow characteristic is associated with a physiological condition, action can be taken. For example, the physiological condition can be monitored and given early treatment.

### CROSS-REFERENCE TO RELATED APPLICATIONS

This application claims the benefit of Brumfield et al., U.S. Provisional Patent Application No. 60/578,174, entitled, “APPARATUS AND METHOD FOR ASSESSING PERIPHERAL CIRCULATION TO EVALUATE A PHYSIOLOGICAL CONDITION,” filed Jun. 8, 2004, which is hereby incorporated by reference herein.

### FIELD

The field relates to the field of medical evaluation, and more specifically, to an apparatus and method for the non-invasive evaluation, detection, and monitoring of a physiological state or medical condition by assessing peripheral circulation.

### BACKGROUND

Technology for detecting hemodynamic events in body extremities has provided significant advances in the field of medicine. Many diagnostic and treatment procedures require an accurate measure of blood flow. The widespread availability of skilled technicians and reduction in cost of the necessary equipment has encouraged the use of monitoring changes in the peripheral arterial vasoconstriction as a part of routine preventive care. A number of techniques now make it possible to routinely assess peripheral circulation within the body. However, translating peripheral circulation measurements into meaningful evaluations of physiological conditions has been difficult.

While it has been known that arterial tone is a mechanism used by the body to control various functioning parameters, changes in arterial tone in response to states or conditions can also be used as a valuable diagnostic tool for physiological conditions. Changes in the peripheral arterial tone may be detected by monitoring changes in any number of hemodynamic parameters such blood flow, blood volume, and the shape of the arterial wave. While changes can be measured in any number of peripheral arteries, such as in the patient's skin, it is commonly conducted on one of the patient's digits (fingers or toes) to detect pulsatile volume of arterial blood of such location. The finger is an advantageous site because of its easy access, but other regions of the body extremity could also be used.

Methods and apparatuses for monitoring blood flow can generally be classified as either non-invasive or invasive. The non-invasive methods are commonly used when periodic individual measurements of arterial systolic and diastolic blood pressure data are sufficient. Invasive methods are used when reliable continuous monitoring of the blood pressure is needed. The conventional method of continuous blood pressure measurement involves the introduction of an intra-arterial cannula which transmits the intra-arterial pressure waveform to a pressure measuring apparatus. Due to its invasiveness, continuous monitoring is usually confined to critical care environments and operating rooms. Non-invasive techniques reduce the risk of observation-related injury or complication and reduce discomfort and inconvenience for the observed patient. These advantages encourage patients to undergo more frequent screening and permit earlier detection of potentially life-threatening conditions. For example, circulatory conditions can be identified and diagnosed at an early stage, when treatment may be more likely to be successful.

The changes in the amount of blood in a peripheral anatomical structure can be determined by any number of non-invasive techniques. The most common methods are auscultatory and oscillometric sphygmomanometric methods, according to which a cuff is placed on the upper arm and is inflated until the artery is completely occluded. Such methods do not always accurately measure peripheral circulation levels due to the location of the reading. An alternate and widely used technique for peripheral blood flow detection is photoplethysmography. Photoplethysmography (hereinafter abbreviated to PPG) has been known to the art for more than fifty years and is applied technically for measuring peripheral blood circulation. A photoplethysmograph consists of a light transmitter and receiver. The transmitter and receiver can be placed on opposite sides of the finger tip, and the receiver records the changing transmission of light through the finger due to the changing amount of blood flowing through the artery. In an alternate embodiment, a photoplethysmograph can have the light transmitter and receiver on the same side of the finger, with the receiver recording the changing reflected light due to the changing amount of blood flowing through the artery. In either embodiment, the received signal is transmitted to a processor for converting the receiver output into diastolic and systolic pressure data. Despite such technologies, there is a need for an improved simple, non-invasive technique for acquiring peripheral blood circulation data.

In another commonly used technique for assessing blood flow changes, called the Prusik-Wallis nail press, a physician visually assesses color return to a nail of a digit following a ten-second finger press to the nail. This technique can provide a quick and reliable test for determining major peripheral circulation problems. Unfortunately, this technique is subjective and cannot provide precise quantitative measures for more accurately assessing peripheral circulation and evaluating physiological conditions. Accordingly, there is a need for an automated nail press test with objective methods for evaluating physiological conditions from the obtained quantitative measures.

Furthermore, although progress has been made in employing software to assist in detection of physiological conditions from changes in peripheral arterial tone, there are significant limitations to the current automated techniques. For example, determining the quality and stability of peripheral blood flow data for use in analysis is one problem consistently plaguing such systems. It is important that the signal used in analysis be representative of the true peripheral arterial tone and not include abnormal fluctuations due to patient movement, patient excitability, or device malfunction. Particular examples of common problems with PPG analysis are the misidentification of stable data and the inaccurate determination of the mean pulse, which can lead to false positive evaluations of physiological conditions. Thus, there is a need for improved computer-based approaches for identifying stable PPG data and determining the mean pulse from resting PPG data.

### SUMMARY

Embodiments described herein include apparatuses, methods and systems for acquiring and assessing peripheral circulation data for evaluating physiological conditions. For example, peripheral blood flow data from a subject can be acquired and then analyzed to determine blood flow characteristics. The blood flow characteristics can then be used to evaluate physiological conditions of the subject.

One embodiment of an apparatus is operable to acquire digital representations of the blood flow of a digit and process the digital representations to determine a signal representative of blood flow. Components in each digital representation can be categorized into groups of components based on light criteria (for example, brightness and spectrum). The groups of components for the digital representations can then be analyzed to determine a blood flow signal.

In another embodiment, an apparatus acquires a blood flow signal (e.g. via a PPG detector and/or via digital representations), measures a force applied to the digit of a subject, processes the measured force and blood flow signal to detect at least one blood flow characteristic, and evaluates a physiological condition of the subject based on characteristics of blood flow.

Blood flow characteristics can be determined from acquired signals representing resting blood flow and signals representing a change in blood flow. The change in blood flow can be due to an applied force or due to a physiological state or condition. The stability of the acquired signals can be analyzed prior to determining characteristics of blood flow in order to reduce false positives in evaluating physiological conditions. A mean pulse can be determined from the signal representing resting blood flow based upon linear associations between pulses in the signal. Blood flow characteristics can be determined from the mean pulse.

The determined characteristics of peripheral blood flow of a subject can then be classified as of interest (e.g., a characteristic associated with a physiological condition requiring further evaluation and consideration of the physiological condition) or not of interest (e.g. a characteristic not associated with a physiological condition and therefore not requiring further evaluation and consideration).

In some embodiments, a set of one or more peripheral blood flow signals is processed via a number of techniques to collect various characteristics of the peripheral blood flow of a subject. A software classifier can use the blood flow characteristics to classify the characteristics (e.g. as of interest or not of interest) to evaluate physiological conditions of the subject.

The characteristics can be used as input to a classifier, such as a rule-based system, a neural network, or a support vector machine. The classifier can draw upon the various characteristics to provide an evaluation of a subject's physiological condition based on a classification of the characteristics (e.g. as being of interest or not being of interest).

The technologies can be applied to any of a variety of physiological conditions, such as conditions demonstrating critical changes in peripheral circulation, including heart disease, peripheral vascular disease, diabetes, Raynaud's phenomenon, and hand-arm vibration syndrome (HAVS). Additionally, the technologies can be applied to conditions such as age and handedness.

Blood flow signals and characteristics of blood flow can be depicted in user interfaces, whether or not a physiological condition is evaluated.

Additional features and advantages of the invention will be made apparent from the following detailed description of illustrated embodiments, which proceeds with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a perspective view of an apparatus, according to one embodiment, that can be used to measure and/or evaluate blood flow in a digit.

FIG. 2 is a perspective view illustrating another embodiment of an apparatus that can be used to measure and/or evaluate blood flow in a digit.

FIG. 3 is a front elevation view illustrating another embodiment of an apparatus that can be used to measure and/or evaluate blood flow in a digit.

FIG. 4 is an enlarged, side elevation view illustrating the apparatus of FIG. 1.

FIG. 5 is a side elevation view illustrating another embodiment of an apparatus that can be used to measure and/or evaluate blood flow in a digit.

FIG. 6 is an elevation view of the apparatus shown in FIG. 5.

FIG. 7 is a perspective view of an apparatus that can be used to measure and/or evaluate blood flow in a digit, according to yet another embodiment.

FIG. 8 is a block diagram of an exemplary system for processing digital representations of blood flow within a feature with software to determine a blood flow signal.

FIG. 9 is a flowchart showing an exemplary method for processing digital representations of blood flow within a feature to determine a blood flow signal.

FIG. 10 is a block diagram of an exemplary system for determining a blood flow signal representative of blood flow within a feature via a plurality of digital representations of blood flow within the feature.

FIG. 11 is a flowchart showing an exemplary method for determining a blood flow signal representative of the blood flow within a feature via a plurality of digital representations of blood flow within the feature.

FIG. 12 is a screen shot of a plurality of digital representations of a digit, including digital representations showing changes in blood flow within the digit.

FIGS. 13A and B are screen shots of digital representations of a digit, including results of an exemplary component classifier applied to the digital representations.

FIG. 14 is a screen shot illustrating the results of an exemplary component classifier applied to a digital representation of a feature.

FIG. 15 is a screen shot illustrating the changes in groups of components in a plurality of digital representations of a feature.

FIG. 16 is a screen shot illustrating blood signals determined from a plurality of classified groups of components from a plurality of digital representations of a feature captured during a time interval.

FIG. 17 is a block diagram of an exemplary system for processing a photoplethysmograph signal of blood flow with software to determine a stable photoplethysmograph signal of the blood blow for use in the evaluation of a physiological condition of a subject.

FIG. 18 is a flowchart showing an exemplary method for determining the stability of a photoplethysmograph signal of blood flow for use in the evaluation of a physiological condition of a subject.

FIG. 19 is a block diagram of an exemplary system for determining the stability of a photoplethysmograph signal of blood flow via current signal stabilizers.

FIG. 20 is a flowchart showing an exemplary method for determining the stability of a photoplethysmograph signal of blood flow via current signal stabilizers.

FIG. 21 is a block diagram of an exemplary system for processing a blood flow signal of a feature with software to determine at least one blood flow characteristic for evaluating a physiological condition of a subject.

FIG. 22 is a flowchart showing an exemplary method for processing a blood flow signal of a feature to determine at least one blood flow characteristic for evaluating a physiological condition of a subject.

FIG. 23 is a block diagram showing an exemplary system for processing a blood flow signal of a feature to evaluate a physiological condition of a subject.

FIG. 24 is a flowchart showing an exemplary method for processing a blood flow signal of a feature to evaluate a physiological condition of a subject.

FIG. 25 is a block diagram of an exemplary system for processing a plurality of blood flow characteristics with software to classify the characteristics to evaluate a physiological condition of a subject.

FIG. 26 is a block diagram of an exemplary system for determining a blood flow characteristic via a mean pulse of a photoplethysmograph blood flow signal.

FIG. 27 is a flowchart showing an exemplary method for determining a blood flow characteristic via a mean pulse of a photoplethysmograph blood flow signal.

FIG. 28 is a flowchart showing another exemplary method for determining a blood flow characteristic via a mean pulse of a photoplethysmograph blood flow signal.

FIG. 29 is a flowchart showing still another exemplary method for determining a blood flow characteristic via a mean pulse of a photoplethysmograph blood flow signal.

FIG. 30 illustrates an exemplary method for determining a mean pulse of a photoplethysmograph blood flow signal.

FIG. 31 illustrates a minimum rise time pulse parameter of a mean pulse.

FIG. 32 illustrates a stiffness index pulse parameter of a mean pulse.

FIG. 33 is a block diagram of an exemplary system for determining a blood flow characteristic via applying a force to a feature so as to cause a change in blood flow in the feature.

FIG. 34 is a flowchart showing an exemplary method for determining a blood flow characteristic via applying a force to a feature so as to cause a change in blood flow in the feature.

FIG. 35 is a flowchart showing another exemplary method for determining a blood flow characteristic via applying a force to a feature so as to cause a change in blood flow in the feature.

FIG. 36 is a screen shot of a graph of force applied to a feature over time so as to cause a change in blood flow in the feature.

FIGS. 37A and 36B are screen shots illustrating rates of changes in blood flow in a feature and force applied to a feature over a time interval.

FIG. 38 is a screen shot illustrating a photoplethysmograph blood flow signal of a feature and a signal representative of the amount of force applied to a feature over a time interval.

FIG. 39 illustrates exemplary force and blood flow signal time points for determining a blood flow characteristic corresponding to a change in blood flow of a feature.

FIG. 40 illustrates photoplethysmograph blood flow signal parameters for determining a blood flow characteristic corresponding to a change in blood flow of a feature.

FIG. 41 is a flowchart showing an exemplary method for evaluating physiological conditions of a subject from peripheral blood flow signals.

FIG. 42 is a graph showing the mean stiffness index (with a standard error of measurement) of 43 subjects, determined from the mean pulse parameters derived from blood flow signals from digits on the left and right hands of the subjects.

FIG. 43 is a graph showing the mean time difference (with a standard error of measurement) between the systolic peak and the diastolic peak of the mean pulses of 43 subjects, derived from blood flow signals from digits on the left and right hands of the subjects.

FIG. 44 is a group of graphs showing mean pulse parameters of 43 subjects, derived from blood flow signals from digits on the left and right hands of the subjects, separated into age groups.

FIG. 45 is a group of graphs showing mean pulse shapes of 43 subjects, derived from blood flow signals from digits on the left and right hands of the subjects, separated into age groups.

FIG. 46 is a graph showing the minimum rise time pulse parameter of multiple subjects, determined from the mean pulse of blood flow signals from digits of the multiple subjects, separated into age groups.

FIG. 47 is a graph showing the minimum rise time pulse parameter of multiple non-smoking, non-High Blood Pressure, and/or non-diabetic subjects, determined from the mean pulse of blood flow signals from digits of the multiple subjects, separated into age groups.

FIG. 48 is a screen shot of an interactive front panel for providing access to a variety of the described technologies.

FIG. 49 is a block diagram of an exemplary computer system for implementing the described technologies.

### DETAILED DESCRIPTION

### Overview of Technologies

The technologies described herein can be used in any of a variety of scenarios in which evaluation of a physiological condition is useful. For example, patient assessment during a patient-medical provider encounter can include a non-invasive evaluation, detection, and monitoring of a physiological state or medical condition by assessing peripheral circulation. This can be useful in that it may permit early detection of potentially life-threatening conditions, as well as regular monitoring of physiological conditions in which changes in the condition could be of concern. For example, circulatory conditions can be identified and diagnosed at an early stage, when treatment may be more likely to be successful.

Automated determination of a peripheral blood flow signal and detection of blood flow characteristics from the blood flow signal can result in a list of candidate blood flow characteristics. The candidate blood flow characteristics can be evaluated to determine whether the candidate blood flow characteristic is of interest or not. If a candidate blood flow characteristic is identified as not of interest, it can be acted upon accordingly (such as the being removed from a list of candidate blood flow characteristics that are associated with a particular physiological condition).

It is important that characteristics of peripheral blood flow in subjects be detected and classified as of interest because such characteristics can enable early detection of physiological conditions in which early treatment is valuable and life-saving. Additionally, determining peripheral blood flow characteristics can be helpful for non-invasively monitoring and evaluating physiological conditions.

### Definitions

A feature includes any anatomical structure or portion of an anatomical structure. For example, a feature can be any peripheral anatomical structure such as a digit, hand, arm, foot, leg, head, ear, nose or any other peripheral anatomical structure found in human beings or other vertebrates. A feature can also include any other anatomical structure or portion thereof found in human beings or other vertebrates in which blood flows.

A digit includes any finger or toe in human beings or corresponding part in other vertebrates.

A digital representation includes any digital representation of a feature stored for processing in a digital computer. For example, digital representations can include two- or three-dimensional representations of portions of an anatomical structure stored as images via a variety of data structures. Representations can be composed of pixels, voxels, or other elements. A digital representation of an anatomical structure is sometimes called “virtual” (for example, a “virtual digit” or a “virtual blood flow”) because it is a digital representation that can be analyzed to learn about the represented anatomical structure(s). A digital representation can be obtained through imaging technologies.

A component of a digital representation includes any two- or three-dimensional element that composes a part of a digital representation of an anatomical structure(s) (or portion thereof). For example, pixels and voxels can be components.

Imaging includes any technique for obtaining one or more digital representations of the body (or portion thereof) by transmitting and/or reflecting light, electromagnetic, or sonic waves through or against the body. Imaging includes the optical counterpart of an object produced by an optical device (e.g. lens or mirror), electronic device (e.g. digital camera), radiographic images (e.g. X-rays in a CT), sonic energy (e.g. ultrasound) and magnetic fields (e.g. MRI).

Photoplethysmography includes any techniques for obtaining a determination of blood volume of a respective area by measuring the intensity of light reflected from the surface of the skin and the red blood cells in the blood below the skin. This can include both transmission and reflectance techniques.

Blood flow includes the movement of blood through a circulatory pathway.

A blood flow measurement includes any measurement of any number of hemodynamic parameters, such as arterial tone, blood volume, the shape of the arterial wave, and the like. For example, blood volume may be expressed in terms of its direct current (DC) component and/or alternating current (AC) component.

A blood flow signal includes any signal that is representative of blood flow. For example, a signal that is representative of one or more blood flow measurements can be a blood flow signal. One such measurement can be the determination of blood volume of a respective area of a feature. Blood flow signals can include resting blood flow signals as well as blood flow signals that represent changes in blood flow.

A blood flow characteristic includes any distinguishing trait, quality, or property of blood flow. Blood flow characteristics can include changes in blood flow (for example, the rate of change and difference between levels of two blood flow measurements), as well as time intervals representative of changes in blood flow. In some cases, the term “parameter” can be used synonymously with “characteristic.”

A blood flow characteristic of interest includes any blood flow characteristic that is of interest in evaluating one or more physiological conditions. In practice, blood flow characteristics of interest can include those blood flow characteristics that require further review by a human review (e.g. a medical practitioner). For example, blood flow characteristics of interest can include characteristics or measurements of characteristics that are associated with physiological conditions, including physiological conditions demonstrating critical changes in peripheral circulation and the like.

In a fully automated system, the characteristics, measurements of characteristics, and physiological conditions associated with the blood flow characteristic of interest can be provided as a result. In a system with user (e.g. health specialist) input and/or assistance, a blood flow characteristic can be presented to the user for confirmation or rejection of the characteristic as being of interest. Those characteristics confirmed as being of interest can then be provided as a result.

A candidate blood flow characteristic of interest includes any blood flow characteristic identified as a possible blood flow characteristic of interest by software. For example, software may preliminarily identify a set of candidate blood flow characteristics of interest (for example, characteristics associated with a physiological condition), some of which can include false positives. Software can then identify the blood flow characteristics of interest within the candidates (for example, by comparing the blood flow characteristics with blood flow characteristics from subjects with specified physiological conditions or by analyzing multiple blood flow characteristics known to be found in combination with one another when associated with a specified physiological condition).

Classifying includes classifying component types, for example designated individual components as members of particular groups of components based on some similarity. For example, components can be classified according to light levels.

Classifying also includes designating a blood flow characteristic as of interest or as not of interest (e.g. disqualifying a blood flow characteristic as being of interest). For example, in the case of a blood flow characteristic determined from a virtual digit, a blood flow characteristic can be classified as of interest because it is associated with a particular physiological condition, thereby associating the subject with the condition.

A stable blood flow signal includes any dependable blood flow signal that is representative of blood flow which represents little or no fluctuation than what is expected from the conditions. For example, blood flow to a digit is expected to maintain a relatively constant equilibrium state under constant conditions, and the signal should correspond to that. However, if a subject were to become temporarily excited during a resting phase, blood flow could become unstable and the signal would represent such instability. Similarly, should equipment that measures blood flow malfunction, readings could be unstable and inaccurately represent the true blood flow.

Valley includes any low point or groups of low points within a blood flow signal at which contraction of the artery has ended and expansion of the artery has not yet begun. For example, on a pulse blood flow signal, valleys can resemble dips.

Peak includes any high point or groups of high points within a pulse in a blood flow signal at which expansion of the artery is at its highest before contraction begins. For example, on a pulse blood flow signal, peaks can resemble rises.

Pulse width includes any determination of the time interval between two points of a pulse in a blood flow signal. For example, in a photoplethysmograph signal of blood flow, pulse width can be determined by the time between two adjacent valleys or dips in the signal.

Pulse area includes any determination of an area defined by a pulse in a blood flow signal. For example, in a photoplethysmograph signal of blood flow, pulse area can be determined by the integral or area beneath the curve represented by the signal between two adjacent valleys or dips in the signal.

Pulse height includes any determination of the height of a pulse in a blood flow signal. For example, in a photoplethysmograph signal of blood flow, pulse height can be determined by the distance between an adjacent valley (or dip) and peak (or rise) in the signal.

Blood volume return includes any increase in blood flow from a level in which blood flow had been reduced.

Rate of change includes any measurement of a value that results from dividing the change in a function of a variable by the change in the variable.

Rate of return of blood flow includes the rate of change of blood volume return. For example, rate of return of blood flow includes determining the rate at which blood flow into a digit increases after a pressing force is removed from the digit.

Return time includes any time interval representative of blood volume return to/towards a baseline blood volume.

A baseline blood volume includes any measurement of blood volume in a feature when normal conditions exist. For example, a baseline blood volume measurement can be the level of a stable blood flow measurement from a digit prior to any force being applied to the digit.

Linear associations includes any relationship between two or more variables in which the variables can be shown to increase or decrease in association with one another to produce a relatively straight line. For example, correlation can be used to measure the level of association as a correlation coefficient to determine how closely the association of two variables resemble a straight line (or one another).

### EXAMPLE 1

### Exemplary Apparatus for Evaluating Blood Flow within a Digit

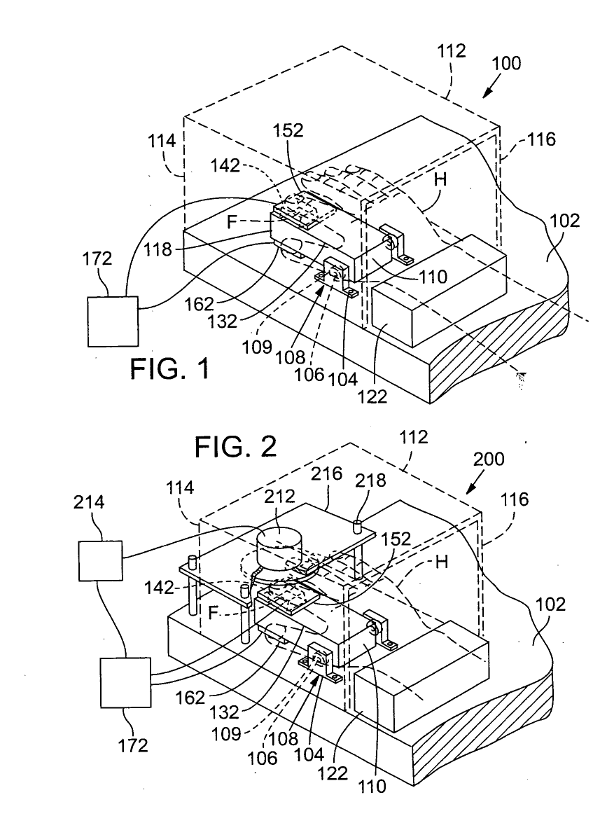

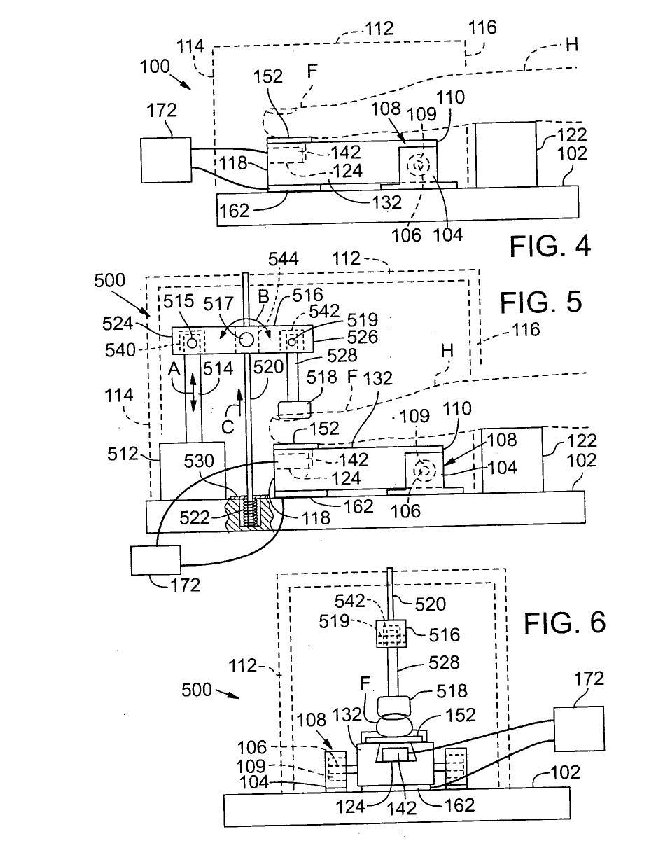

FIGS. 1 and 4 illustrate an apparatus 100, according to one embodiment, that can be used to detect, measure, and/or evaluate blood flow within a digit. In the illustrated embodiment, apparatus 100 includes a cover 112 supported on a base or support 102. Cover 112 can be closed or open at one end 114 and open at the opposite end 116 for receiving a hand H. A hand rest 122 is located on base 102 to provide a surface for resting the wrist or distal end of the arm. A pivotable lever 132 located proximate to hand rest 122 is adapted to receive a finger F from which blood flow can be evaluated. A force measuring device 162 is positioned underneath the distal end 118 of lever 132 to measure the downward force exerted on lever 132 by the finger F. In other embodiments, instead of a lever, a stationary surface can be used for receiving finger F from which blood flow can be evaluated.

Lever 132 is mounted for pivoting movement relative to the base 102, such as with the illustrated hinge assembly 108. Hinge assembly 108 in the illustrated configuration includes upright brackets 104 mounted to base 102 on opposite sides of the proximal end 110 of lever 132. A bearing 109 can be housed in each bracket 104, such as via an interference fit. A pin 106 extends through bearings 109 and the lever proximal end 110 so as to allow lever 132 to pivot upwardly and downwardly relative to the base. This concentrates the force exerted by finger F at the distal end 118 of lever 132 for more accurate force measurements.

Apparatus 100 includes a blood flow detector for detecting and/or measuring blood flow within the finger F. In particular embodiments, the blood flow detector is a photoplethysmograph detector 142 embedded or placed within a recess 124 formed in the distal end 118 of lever 132. The photoplethysmograph detector 142 is configured to detect blood flow and generate a blood flow signal representative of the blood flow in the finger F. The photoplethysmograph detector includes an infrared light source (not shown) (for example, an LED) for directing light at finger F and a light detector (not shown) for measuring the amount of infrared light reflected back from the finger F and generating a signal representative of blood flow.

In the illustrated embodiment, lever 132 further includes an infra-red transparent cover or plate 152 (for example, a cold mirror) located above the photoplethysmograph detector 142. Cover 152 reflects short wavelengths of light (for example, visible light) and transmits long wavelengths of light (for example, infra-red light), thereby reducing ambient light from reaching the light detector and increasing the accuracy of the blood flow reading.

In other embodiments, instead of a reflective photoplethysmograph, a transmissive photoplethysmograph which includes a light detector above finger F can be used to measure the blood flow through finger F. The transmissive photoplethysmograph measures the amount of light transmitted through finger F to generate a blood flow signal representative of the blood flow in finger F. Other types of blood flow detectors can also be used (for example, a Cadmium-Telluride detector used in combination with administering radioactive compounds into a subject's blood stream or an Ultrasonic Doppler device).

Force measuring device 162 is used to measure the downward force exerted on lever 142 by the finger F. Force measuring device 162 can be, for example, a load cell, a load cell coupled with a strain gauge amplifier, or any equivalent mechanism. Blood flow detector 142 and force measuring device 162 in the illustrated embodiment are operably coupled to a signal processor 172 using suitable techniques (for example, a hard wired connection or any of various wireless technologies). In use, the user presses the distal end of the finger F downwardly against the lever 132 to cause a change in blood flow in that portion of the finger. Blood flow detector 142 and force measuring device 162 detect the blood flow and the applied force and send respective signals representative of blood flow and force to signal processor 172. Signal processor 172 receives and processes the signals from blood flow detector 142 and force measuring device 162. In certain embodiments, for example, the signal processor generates one or more blood flow characteristics based on one or more of these signals, and evaluates one or more physiological conditions of the user based on such blood flow characteristics.

Apparatus 100 (and other apparatuses described herein) also may include a monitor or other visual display to display the signals and/or data processed by the signal processor. In certain embodiments, apparatus 100 (and other apparatuses described herein) can include a graphical user interface program that can display acquired signals and other data and allows a user to interface with the apparatus, such as described in the examples below.

### EXAMPLE 2

### Exemplary Apparatus for Evaluating Blood Flow within a Digit

FIG. 2 illustrates an apparatus 200, according to one embodiment, that can be used to detect, measure, and/or evaluate blood flow within a digit. Components in the embodiment shown in FIGS. 1 and 4 which are similar to components in apparatus 200 have the same respective numerals and therefore are not further described.

Apparatus 200 includes a camera 212 for acquiring digital representations of the blood flow within finger F. In other embodiments, other imaging techniques can be used to acquire digital representations of the blood flow. In the illustrated embodiment, camera 212 is supported above lever 132 on camera support 216, which is supported above base 102 by posts 218. In other embodiments, camera 212 can be located in front of the finger F, rather than above the finger, to acquire digital representations of the distal tip of finger F. Camera 212 is operably coupled to a digital representation processor 214 using suitable techniques (for example, a hard wired connection or any of various wireless technologies). A digital representation processor 214 is operatively coupled to a signal processor 172 using suitable techniques (for example, a hard wired connection or any of various wireless technologies). Digital representation processor 214 is configured to receive digital representations of blood flow from camera 212 and generate a signal representative of the blood flow in finger F. In certain embodiments, digital representation processor 214 and signal processor 172 can be combined into a single processor.

In use, the user presses the distal end of the finger F downwardly against the lever 132 to cause a change in blood flow in that portion of the finger. Camera 214 acquires digital representations of the blood flow and sends the digital representations to digital representation processor 214. Digital representation processor 214 generates a signal representative of the blood flow in finger F and sends the signal to a signal processor 172. In some embodiments, as illustrated, a blood flow detector 142 can also be used to detect the blood flow and send a signal representative of blood flow to signal processor 172. A force measuring device 162 detects the applied force and sends a respective signal representative of the applied force to signal processor 172. Signal processor 172 receives and processes the signals from digital representation processor 214, and/or blood flow detector 142, and force measuring device 162. In certain embodiments, for example, the signal processor generates one or more blood flow characteristics based on one or more of these signals, and evaluates one or more physiological conditions of the user based on such blood flow characteristics.

### EXAMPLE 3

### Exemplary Apparatus for Evaluating Blood Flow within a Digit

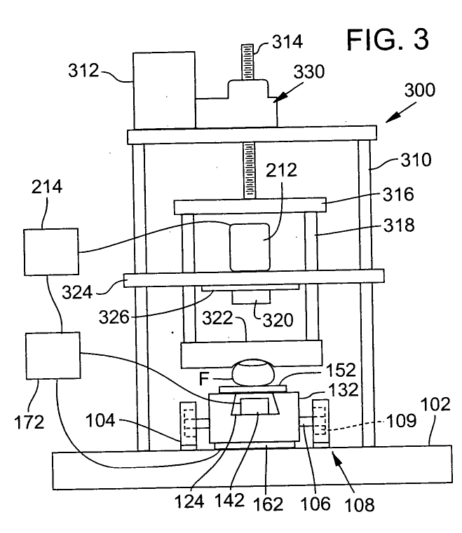

FIG. 3 illustrates an apparatus 300, according to another embodiment, that can be used to detect, measure, and/or evaluate blood flow within a digit. Apparatus 300 combines features of apparatus 100 shown in FIGS. 1 and 4 and apparatus 200 shown in FIG. 2. Components of the embodiments shown in FIGS. 1, 2, and 4 which are similar to components in apparatus 300 have the same respective numerals and therefore are not further described.

Apparatus 300 includes a motor 312 operatively coupled to a pressing plate 322. Motor 312 is operable to cause the pressing plate 322 to move downwardly against finger F to apply a gradually increasing force and to move upwardly away from finger F to decrease or remove the force. In the illustrated embodiment, motor 312 is mounted on a cover 310 and is operatively coupled to a drive transmission mechanism 330. Drive transmission mechanism 330 is operatively coupled to a screw 314, which extends downwardly through at corresponding opening in cover 310. The lower end of screw 314 is coupled to a plate 316, which in turn is coupled to rods 318. Rods 318 extend downwardly through corresponding openings in a camera support 324, which supports a camera 212. Lens 320 of the camera 212 extends downwardly through at corresponding opening in camera support 324. A focus wheel 326 is operatively coupled to lens 320. In other embodiments, camera 212 can be located in front of finger F, rather than above the finger, to acquire digital representations of the distal tip of finger F. The lower end of rods 318 are coupled to pressing plate 322. Pressing plate 322 is transparent to allow camera 212 to capture images of finger F.

In use, motor 312 rotates screw 314 to cause pressing plate 322 to move downwardly and press against the distal end of the finger F. Finger F correspondingly presses downwardly against the lever 132 to cause a change in blood flow in that portion of the finger. After the force is applied to finger F for a desired period of time, the screw 314 is rotated in the opposite direction to move pressing plate 322 upwardly to reduce or remove the force from the finger F. Advantageously, the use of a motor can lead to more consistent and accurate application of a known force compared to a system in which the user presses the finger against a surface to cause a change in blood flow. Motor 312 can include a safety feedback mechanism, such as automatic shutoff or the like, that shuts off the motor or reverses the rotation of the motor should the force measuring device 162 record a force that exceeds a determined safe level.

Camera 212 acquires digital representations of the blood flow and sends the digital representations to a digital representation processor 214. Digital representation processor 214 generates a signal representative of the blood flow in finger F and sends the signal to a signal processor 172. In some embodiments, as illustrated, a blood flow detector 142 can also be used to detect the blood flow and send the signals representative of blood flow to signal processor 172. Force measuring device 162 detects the applied force and sends a signal representative of force to signal processor 172. Signal processor 172 receives and processes the signals from digital representation processor 214, and/or blood flow detector 142, and force measuring device 162. In certain embodiments, for example, the signal processor generates one or more blood flow characteristics based on one or more of these signals, and evaluates one or more physiological conditions of the user based on such blood flow characteristics.

### EXAMPLE 4

### Exemplary Apparatus for Evaluating Blood Flow within a Digit

FIGS. 5 and 6 illustrate an apparatus 500, according to yet another embodiment, that can be used to detect, measure, and/or evaluate blood flow within a digit. Apparatus 500 combines features of apparatus 100 shown in FIGS. 1 and 4 and apparatus 200 shown in FIG. 2. Components of the embodiments shown in FIGS. 1, 2, and 4 which are similar to components in apparatus 500 have the same respective numerals and therefore are not further described.

Apparatus 500 includes a motor 512 (which can be, for example, a stepper motor) coupled to a pressing plate 518. Motor 512 is operable to cause the pressing plate 518 to move downwardly against finger F to apply a gradually increasing force and to move upwardly away from finger F to decrease or remove the force.

More specifically, in the illustrated embodiment, motor 512 is mounted on base 102 and has an upwardly extending shaft 514. Motor 512 is operable to move shaft 514 upwardly and downwardly, as indicated by double-headed arrow A. The upper portion of shaft 514 extends into an enlarged opening 540 in a lever 516 and is pivotably coupled to the lever, such as via a pin 515 that extends through the lever distal end portion 524 and shaft 514. Opening 540 is sized to permit pivoting of the lever relative to the shaft about pin 515.

Lever 516 is pivotably coupled to the upper end portion of a rod or post 528, such as via a pin 519 that extends through the lever proximal end portion 526 and the upper end portion of rod 528. The upper end portion of rod 528 is received in an enlarged opening 542 formed in the lever proximal end portion 526 that is sized to permit pivoting of the lever relative to the rod about pin 519. The lower end of rod 528 is connected to a pressing plate 518.

Lever 516 also pivots relative to a pin 517 (as indicated by double headed arrow B) which extends transversely through the center of lever 516. An upright rod or post 520 extends from base 102 through pin 517 and cover 112. Rod 520 is connected to pin 517 at a fixed location, such as via an interference fit between the rod and the pin. Rod 520 extends through an enlarged, centrally disposed opening 544 extending the height of the lever. Opening 544 is sized to permit pivoting of lever 514 relative to rod 520 about pin 517.

A compression spring 522, which can be embedded in base 102 and capped by a spring cover 530, is disposed on the lower end portion of rod 520. The lower end of spring 522 is attached to the lower end portion of rod 520 and the upper end of spring 522 abuts spring cover 530, but is not attached to the rod, allowing the spring to compress by upward movement of rod 520.

In some embodiments, a camera (not shown in FIG. 5) can be located at any convenient position to acquire digital representations of the blood flow. The camera can be used to send digital representations to a digital representation processor (not shown) which generates a signal representative of the blood flow in finger F and sends the signal to a signal processor 172.

In use, motor 512 is operable to move rod 514 upwardly so as to cause the lever distal end portion 524 to move upwardly, which in turn causes the lever proximal end portion 526 and rod 528 to move downwardly. Downward movement of rod 528 causes pressing plate 518 to press against the distal end of the finger F. Finger F correspondingly presses downwardly against the lever 132 to cause a change in blood flow in that portion of the finger. After the force is applied to finger F for a desired period of time, motor 512 is operated to lower rod 514, which in turn moves pressing plate 518 upwardly to reduce or remove the force from the finger F.

Compression spring 522 serves as a passive mechanical safety mechanism should the motor 512 malfunction (for example, due to software control, circuitry failure, or the like) and apply force that exceeds a predetermined safe level. Compression spring 522 is selected to resist upward movement of rod 520 if the force applied to the finger F is below the predetermined safe level. Should motor 512 apply a force that exceeds the safe force level, rod 520 overcomes the resistance of spring 522 against spring cover 530 and moves upwardly, as indicated by arrow C, thereby allowing lever 516 to also move upwardly and prevent the application of additional force to the finger F.

### EXAMPLE 5

### Exemplary Apparatus for Evaluating Blood Flow within a Digit

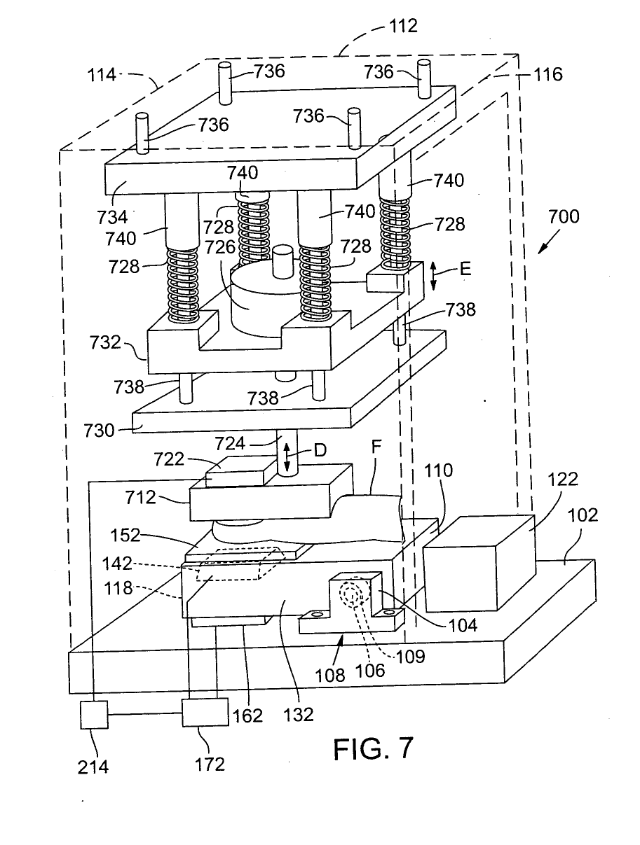

FIG. 7 illustrates an apparatus 700, according to yet another embodiment, that can be used to detect, measure, and/or evaluate blood flow within a digit. Apparatus 700 combines features of apparatus 100 shown in FIGS. 1 and 4 and apparatus 200 shown in FIG. 2. Components of the embodiments shown in FIGS. 1, 2, and 4 which are similar to components in apparatus 700 have the same respective numerals and therefore are not further described.

Apparatus 700 in the illustrated embodiment includes a motor 726 (which can be, for example, a stepper motor) operatively coupled to a pressing plate 712. Motor 726 is operable to cause a pressing plate 712 to move downwardly against finger F to apply a gradually increasing force and to move upwardly away from finger F to decrease or remove the force. Motor 726 in the illustrated embodiment is mounted on a motor-support plate 732 and has a downwardly extending shaft 724 which extends through corresponding openings in motor-support plate 732 and a lower plate 730. The lower end of shaft 724 is secured to pressing plate 712. Motor-support plate 732 is coupled to an upper plate 734 by springs 728, which extend upwardly from the motor-support plate 732 and are secured to respective spacers 740 at the upper ends thereof (such as shown in the illustrated embodiment). Motor-support plate 732 can rest on lower plate 730 or can be suspended by springs 728 (such as shown in the illustrated embodiment), depending on the size of spacers 740 and springs 728. Upper plate 734 is mounted to cover 112, such as by downwardly extending posts 736. Rods or posts 738 extend through springs 728 and motor-support plate 732. The upper end of each rod 738 is connected to the upper plate 734 and the lower end of each rod 738 is connected to the lower plate 730. Motor-support plate 732 is moveable upwardly and downwardly relative to rods 738 and lower plate 730 (as indicated by double-headed arrow E).

A camera 722 can be mounted on pressing plate 712. Pressing plate 712 is transparent to allow camera 722 to capture images of finger F. In other embodiments, camera 722 can be located in front of finger F, rather than above the finger, to acquire digital representations of the distal tip of finger F.

To apply a force to finger F, motor 726 moves shaft 724 downwardly to cause pressing plate 712 to move downwardly and press against the distal end of the finger F. Finger F correspondingly presses downwardly against the lever 132 to cause a change in blood flow in that portion of the finger. After the force is applied to finger F for a desired period of time, motor 726 is operated to move shaft 724 upwardly to cause pressing plate 712 to move upwardly to reduce or remove the force from the finger F.

Springs 728 serve as a passive mechanical safety mechanism should motor 726 malfunction (for example, due to software control, circuitry failure, or the like) and apply a force that exceeds a predetermined safe level. Springs 728 are selected to resist upward movement of motor-support plate 730 if the downward force applied by pressing plate 712 is less than the predetermined safe force level. Should motor 726 apply a downward force that exceeds the safe force level, motor-support plate 732 will overcome the resistance of springs 728 and will move upward to prevent the application of additional force to finger F by pressing plate 712.

Camera 722 acquires digital representations of the blood flow and sends the digital representations to a digital representation processor 214. Digital representation processor 214 generates a signal representative of the blood flow in finger F and sends the signal to a signal processor 172. In some embodiments, as illustrated, a blood flow detector 142 can also be used to detect the blood flow and send signals representative of blood flow to signal processor 172.

Force measuring device 162 detects the applied force and sends a respective signal representative of force to signal processor 172. Signal processor 172 receives and processes the signals from digital representation processor 214, and/or blood flow detector 142, and force measuring device 162. In certain embodiments, for example, the signal processor generates one or more blood flow characteristics based on one or more of these signals, and evaluates one or more physiological conditions of the user based on such blood flow characteristics.

### EXAMPLE 6

### Exemplary System for Determining a Blood Flow Signal in Digital Representations of Blood Flow

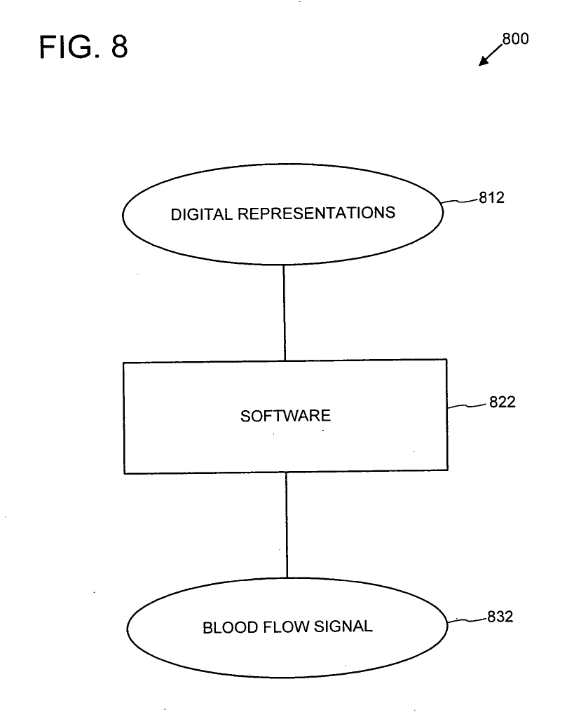

FIG. 8 shows an exemplary system 800 for determining a blood flow signal 832 of a feature from a plurality of digital representations 812 of the feature (for example, the distal end portion of a finger). Apparatus 200 of FIG. 2, apparatus 300 of FIG. 3, or apparatus 700 of FIG. 7, for example, can be implemented to include system 800.

The digital representations 812 (for example, digital images captured by a digital camera) are processed by software 822. Software 822 determines a blood flow signal 832 representative of blood flow in the feature. The software 822 can employ any combination of the technologies described herein.

In any of the examples described herein, a variety of blood flow characteristics can be determined via blood flow signal 832 if desired. For example, mean pulse parameter characteristics and blood flow characteristics based on the change in blood flow can be determined via the blood flow signal. Methods for determining blood flow characteristics are described in detail below.

Further, blood flow signal 832 can be depicted via user interfaces. For example, a graphical depiction of the blood signal can be displayed to a human classifier, who decides what action, if any, to take. Such user interfaces can allow manipulation of the graphical depiction, such as rotating, zooming, and the like.

### EXAMPLE 7

### Exemplary Method for Determining a Blood Flow Signal in Digital Representations of Blood Flow

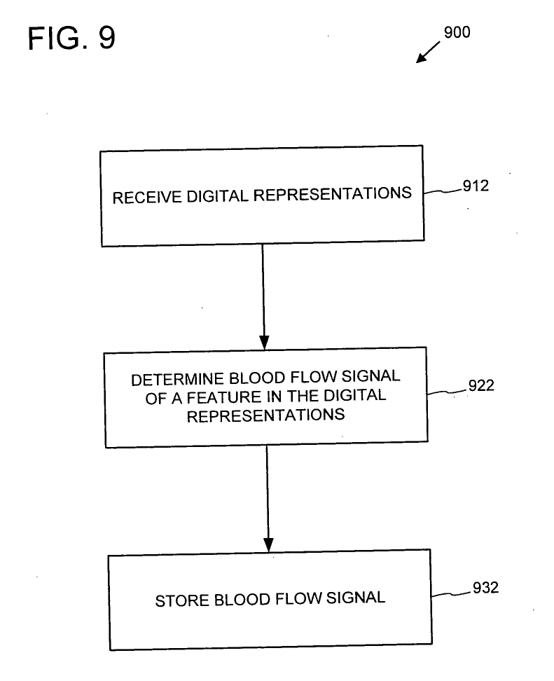

FIG. 9 shows an exemplary method 900 for determining a blood flow signal of a feature represented in a plurality of digital representations. The method can be performed, for example, by system 800 of FIG. 8. Method 900 and any of the other methods described herein can be performed by computer-executable instructions stored on one or more computer-readable media.

At 912, a plurality of digital representations (e.g., the digital representations 812 of FIG. 8) representing a feature are received.

At 922, a blood flow signal of the feature is determined based on the digital representations. As described in the examples, a variety of techniques can be used for determining a blood flow signal. For example, groups of components in the digital representations can be determined, and a signal can be determined based on the grouped components.

At 932, the blood flow signal of the feature can be stored in one or more computer-readable media.

### EXAMPLE 8

### Exemplary System for Determining a Blood Flow Signal Via Grouped Components of Digital Representations

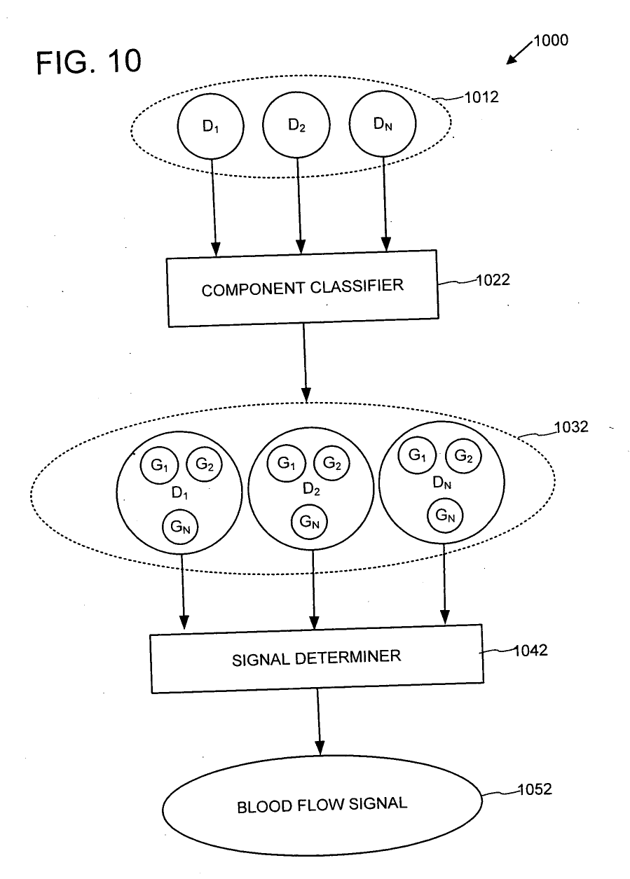

FIG. 10 shows an exemplary system 1000 for determining a blood flow signal of a feature via grouped components of digital representations. The illustrated system 1000 includes a component classifier 1022 and a signal determiner 1042. Component classifier 1022 receives a plurality of digital representations (D₁, D₂, D_{N}) 1012 (for example, the digital representations 812 of FIG. 8) and classifies components (e.g. pixels) of the digital representations into respective groups (G₁, G₂, G_{N}) 1032. The groups of components can be, for example, light levels such as brightness or spectrum. For example, a component that has the same or similar light level as a designated light level group is classified as a member of that respective group.

Signal determiner 1042 receives the groups of classified components 1032 and determines a blood flow signal 1052 of the feature. In one implementation, for example, the number of classified components in group G₁ of digital representation D₁ can be compared to the number of classified components in group G₁ of digital representation D₂, to determine a change in light levels between the two images. This comparison between groups G_{N} can be done for a number of digital representations D_{N} to determine changes in light levels over time. The changes in light levels can then be used to generate a signal representative of blood volume changes.

### EXAMPLE 9

### Exemplary Method for Determining a Blood Flow Signal Via Grouped Components of Digital Representations

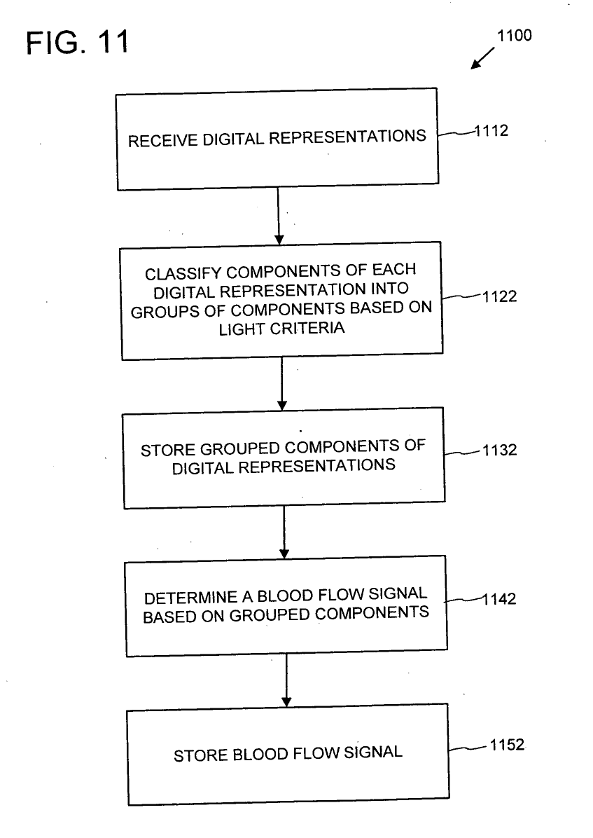

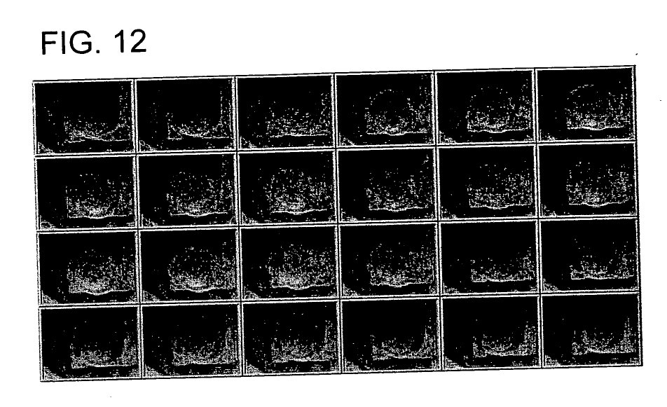

FIG. 11 shows an exemplary method for determining a blood flow signal of a feature via grouped components of digital representations. The method 1100 can be performed, for example, by the system 1000 of FIG. 10.

At 1112, a plurality of digital representations (for example, the digital representations 812 of FIG. 8) representing at least one feature are received. For example, FIG. 12 shows a screen shot of multiple digital representations of the distal end portion of a digit. The digital representations show changes in blood flow within the digit over a time interval during which a force is applied and then removed from the digit, starting with the top left digital representation, proceeding left to right across each row, and ending with the bottom right digital representation. The top left digital representation shows stable, baseline blood flow within the digit prior to applying the force. The bottom right digital representation shows blood flow within the digit after the force has been removed and blood flow within the digit has returned to baseline levels of blood flow or greater.

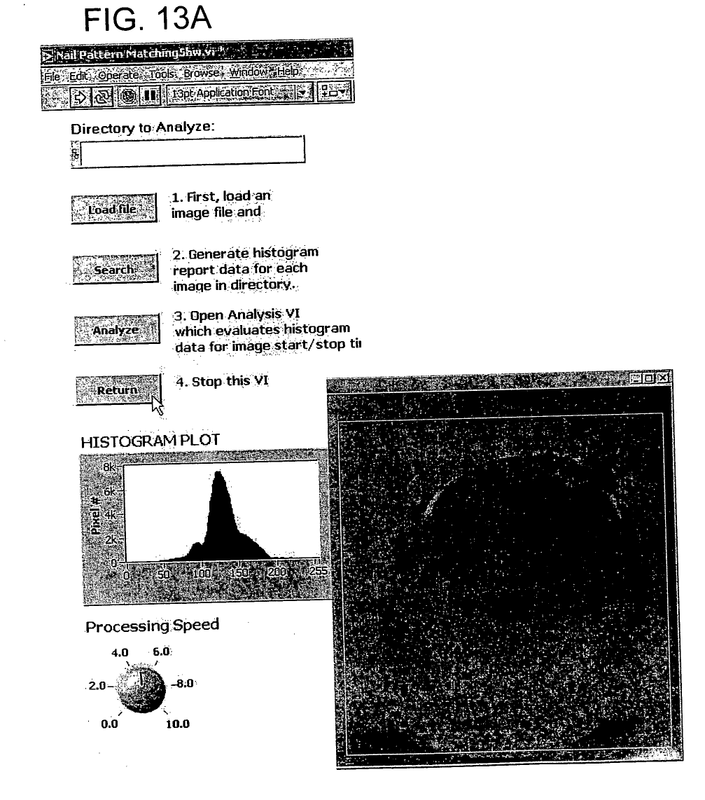

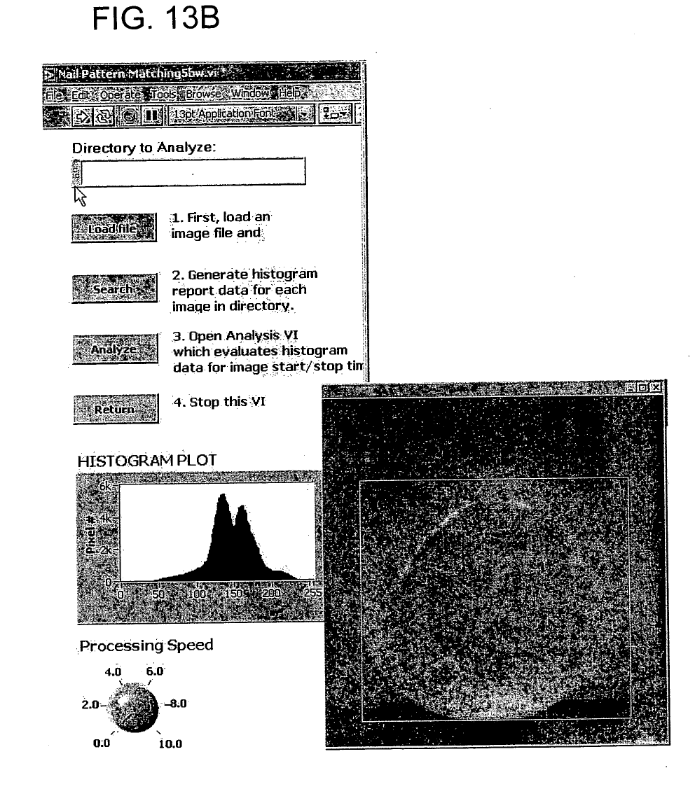

At 1122, components of each digital representation are classified into groups of components based on light criteria. For example, components can be classified according to spectrum or brightness levels. Enlarged screen shots of two of the digital representations of FIG. 12 are shown in FIGS. 13A and B. The screen shots of FIGS. 13A and 13B include exemplary depictions of classified components of the displayed digital representations in the form of histogram plots.

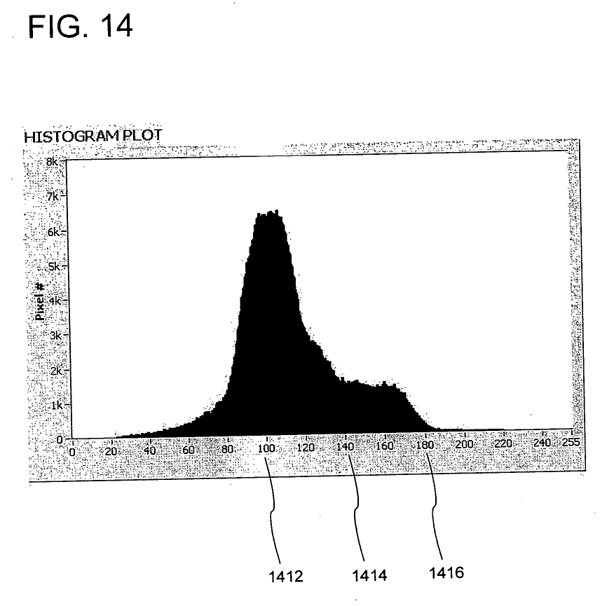

FIG. 14 is an enlarged view of another example of the same type of histogram plot shown in FIGS. 13A and 13B. The histogram plot illustrates each pixel of the corresponding digital representation classified into one of a plurality of groups. Each group (or alternatively referred to as a bin) represents a point on the spectrum from 0-255 in grayscale (black to white). For example, 1412, 1414, 1416 represent specific spectrum groups 100 (G₁₀₀), 140 (G₁₄₀) and 180 (G₁₈₀), and the number of pixels classified in each respective group is shown in the histogram. A plurality of such histogram plots can be generated for respective digital representations of the digit acquired over a time interval. For example, the histograms of respective digital representations would show more pixels classified into the whiter spectrum groups (closer to 255) as the digit becomes whiter (increasingly bloodless) in response to an applied force. Such a change can be seen between FIGS. 13A and 13B, where FIG. 13A represents a digital image captured before the image shown in FIG. 13B.

In other embodiments, groups can represent other qualities or characteristics of the digital representation, such brightness or intensity levels. In still other embodiments, RGB digital representations can be used, in lieu of grayscale, and groups can represent points in the RGB color spectrum.

At 1132, the groups of classified components of the digital representations are stored. The components that are classified into groups (G₁, G₂, G_{N}) for each digital representation (D₁, D₂, D_{N}) are stored. For example, the number of pixels classified into spectrum group 1 for the first digital representation can be stored as G₁D₁, and the number of pixels classified into spectrum group 1 for the second digital representation can be stored as G₁D₂, and so on.

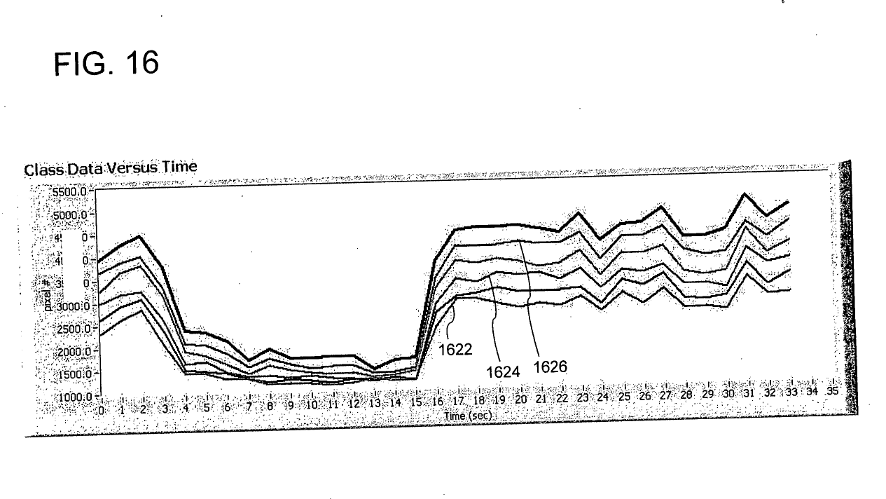

At 1142, a blood flow signal is determined based on the change between at least one group of components in the digital representations. In one implementation, for example, a histogram plot for each spectrum group is generated that illustrates the stored number of components classified into that spectrum group for a plurality of digital representations captured during a time period. For example, a plot for spectrum group 1 (G₁D₁, G₁D₂, to G₁D_{N}) can be a line graph showing the number of pixels in group 1 for each digital representation. The histogram plots show the changes in the number of components in the different spectrum groups over time. In some cases, these plots can be representative of blood flow. FIG. 16, for example, illustrates such a histogram plot for six spectrum groups that display significant changes in the number of classified components in response to an applied force. Such significant changes represent the change in blood volume in response to an applied force and therefore can be representative of blood flow.

While any number of groups can be used to determine a blood flow signal, selecting the spectrum groups that depict the greatest change in the number of components classified into each group over a time period can increase the accuracy of the determined blood flow signal. In order to select the groups with the greatest changes, point-to-point slopes are calculated over each “bin” from each histogram plot of a spectrum group. For example, point-to-point slopes are calculated over spectrum groups 0-255 by determining the slopes between time points 0 and 1, 1 and 2, 2 and 3 and so on for each spectrum group. Typically, the maximum slope for each spectrum group occurs at or about the same time, such as upon the application of force to the finger or upon the release of force applied to the finger. The bin (or spectrum group) with the maximum slope (greatest point-to-point slope) can be chosen, or alternatively the bin with the maximum slope can be chosen along with neighboring bins on either side (for example, three neighboring bins). The chosen bin or bins represent the area of maximal pixel value (or color) change which corresponds to changes in blood flow due to the force application or reduction.

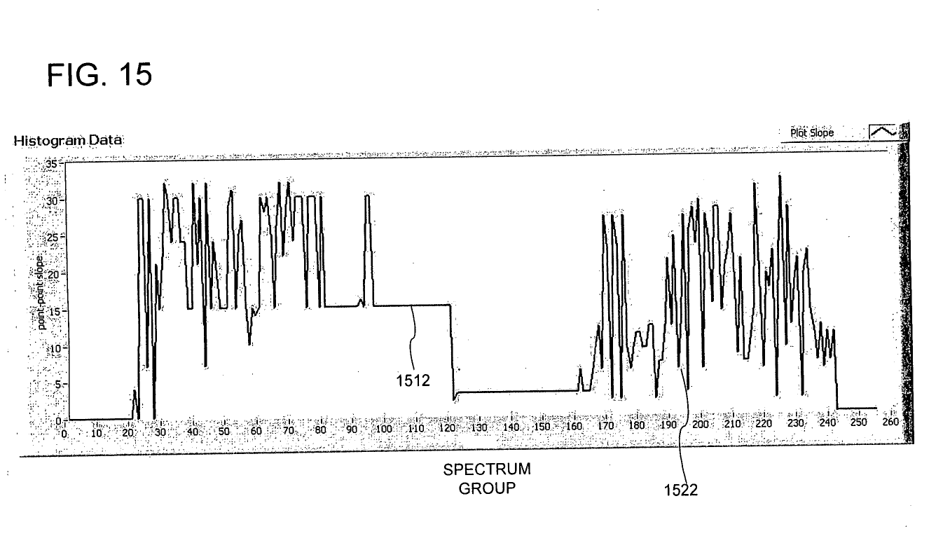

To further improve accuracy, the maximum point-to-point slopes for the spectrum groups can be compared with each other and one or more spectrum groups are selected based on this overall comparison. FIG. 15, for example, illustrates the maximum point-to-point slope for each of the spectrum groups 0-255 in the digital representations shown in FIG. 12. Spectrum group G₁₁₀, indicated at 1512, for example, is in the middle of a plateau representing an area of maximal pixel value change over several “bins.” Such a plateau represents significant color changes over a significant area of the finger. Alternatively, spikes in point-point slopes of spectrum groups (such as spectrum group G₁₉₅, indicated at 1522, and neighboring groups) represent transient, instantaneous changes in color which are more likely to be movement or background related. By choosing an area of the plateau (a group of spectrum groups with similar maximal point-point slopes) to determine a blood flow signal, small areas of color change and artifacts due to movement or background changes can be negated. Spectrum groups can be selected from a plateau in a point-point slope representation, such as shown in FIG. 15 (for example, spectrum group G₁₁₀ and neighboring groups), and distinct changes in pixel classifications are representative of blood flow changes.

FIG. 16 is a histogram plot that illustrates the number of pixels classified into selected spectrum groups (three of which are indicated at 1622, 1624, 1626) that exhibited large changes in the number of classified components for a plurality of digital representations acquired over a time interval. The line for the group with the greatest change in the number of components can be chosen as a representative blood flow signal. In other embodiments, the lines can be combined in any manner to produce an average blood flow signal or any other distinct line can be chosen as the most accurate representative blood flow signal. Alternatively, multiple blood flow signals can be selected from the selected spectrum group line graphs (such as shown in FIG. 16).

At 1152, the determined blood flow signal is stored.

### EXAMPLE 10

### Exemplary Acquisition of Digital Representations

A variety of techniques can be used to acquire digital representations for use with the technologies described herein. In practice, digital representations of an anatomical structure can be acquired; plural digital representations of portions of the anatomical structure can then be extracted therefrom, if desired.

Acquisition of such digital representations is typically done via an optical camera device. However, a scan of the soft tissues of the subject can also be performed. For example, a CT scan can be performed according to any number of standard protocols. CT scans can be used to generate thin-section CT data (for example, helical scan CT data). The representation can be analyzed immediately after the scan, or the representation can be stored for later retrieval and analysis.

Any number of hardware implementations can be used to acquire a representation of an anatomical structure. For example, a high speed CMOS (complementary metal-oxide semiconductor) camera can be used. Any digital camera utilizing CMOS chips, for example chips from Micron Semiconductor Products, Inc., San Jose, Calif. or any other company, can be used. Digital cameras can also utilize any other technology for obtaining high quality digital images. More traditional film cameras can be used as well, with the images converted to digital format via a digital scanner or the like. If CT scans are acquired, the GE HiSpeed Advantage scanner of GE Medical Systems, Milwaukee can be used. Although images for determining blood flow signals in features can be acquired via optical devices, digital camera technology as well as computed tomography imaging (“CT scan”) technology, magnetic resonance imaging (“MRI”) or other imaging technology can be used.

### EXAMPLE 11

### Exemplary System for Determining a Stable Photoplethysmograph Blood Flow Signal

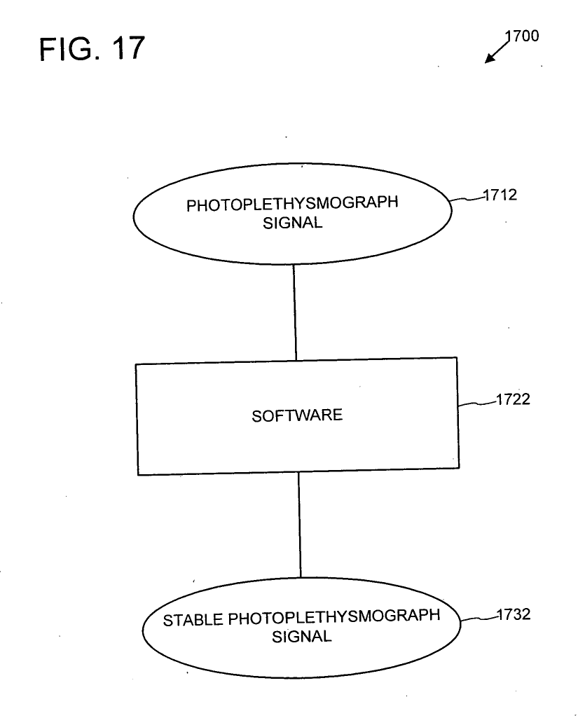

FIG. 17 shows an exemplary system 1700 for determining a stable photoplethysmograph blood flow signal 1732 from a photoplethysmograph blood flow signal 1712 acquired from a feature.

The photoplethysmograph signal 1712 is processed by software 1722 to determine a stable blood flow signal 1732 of the blood flow of the feature. The software 1722 can employ any combination of the technologies described herein.

In any of the examples described herein, a variety of blood flow characteristics can be determined via the stable photoplethysmograph blood flow signal 1732 if desired. For example, mean pulse parameter characteristics and blood flow characteristics based on the change in blood flow can be determined via the stable blood flow signal, as further described below.

Further, the stable blood flow signal 1732 can be depicted via user interfaces.

For example, a graphical depiction of the stable blood flow signal can be displayed to a human classifier, who decides what action, if any, to take. Such user interfaces can allow for the manipulation and selection of sections of the stable photoplethysmograph blood flow signal for use in the evaluation of physiological conditions of the subject.

### EXAMPLE 12

### Exemplary Method for Determining a Stable Photoplethysmograph Blood Flow Signal

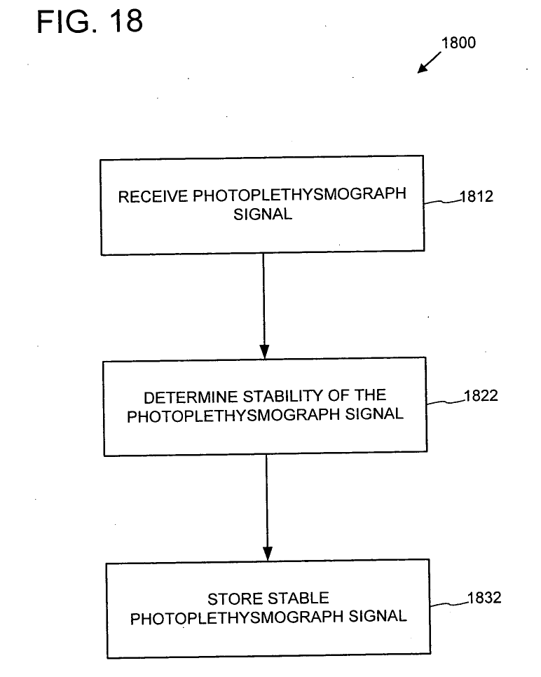

FIG. 18 shows an exemplary method 1800 for determining a stable photoplethysmograph blood flow signal from a photoplethysmograph blood flow signal acquired from a feature. The method can be performed, for example, by system 1700 of FIG. 17. The method 1800 and any of the other methods described herein can be performed by computer-executable instructions stored on one or more computer-readable media.

At 1812, a photoplethysmograph signal (e.g., the photoplethysmograph signal 1712 of FIG. 17) representing the blood flow in a feature is received.

At 1822, the stability of the photoplethysmograph signal of the feature is determined. As described in the examples below, a variety of techniques can be used for determining the stability of the photoplethysmograph blood flow signal. For example, the stability of the current component signals (for example, the direct and alternating current components) in the photoplethysmograph signal can be determined, and a stable signal can be based on the current component stabilities.

At 1832, the stable photoplethysmograph blood flow signal of the feature can be stored in one or more computer-readable media.

### EXAMPLE 13

### Exemplary System for Determining a Stable Photoplethysmograph Blood Flow Signal via Current Signal Stabilizers

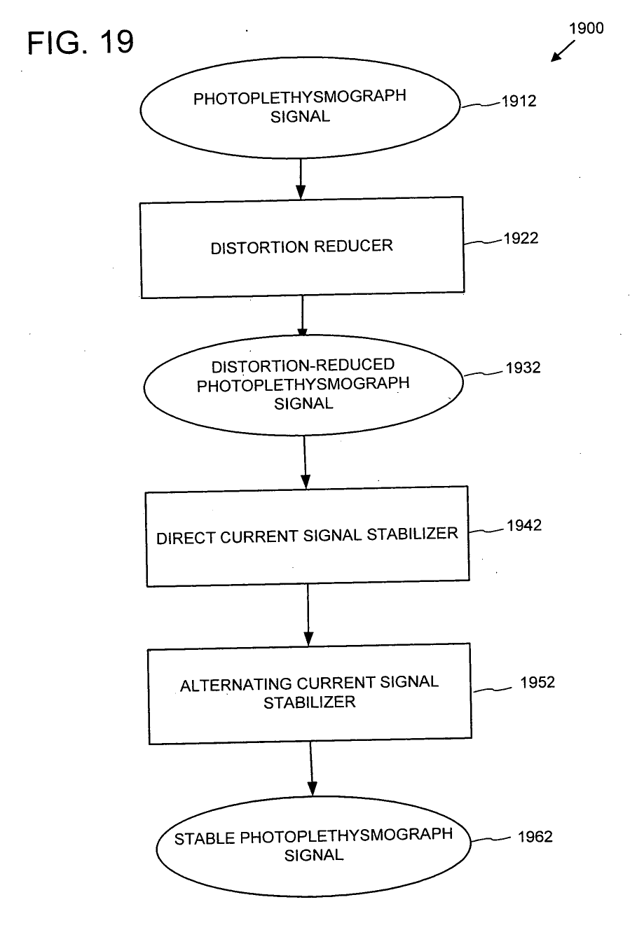

FIG. 19 shows an exemplary system 1900 for determining a stable photoplethysmograph blood flow signal of a feature via current signal stabilizers. A distortion reducer 1922 can receive a photoplethysmograph signal 1912 (for example, the photoplethysmograph signal 1712 of FIG. 17) and determine a distortion-reduced photoplethysmograph signal 1932. A direct current signal stabilizer 1942 and an alternating current stabilizer 1952 can then receive the distortion-reduced photoplethysmograph signal 1932 and determine a stable photoplethysmograph signal. The direct current signal stabilizer 1942 and the alternating current signal stabilizer 1952 can be used in combination in any order or separately to determine a stable photoplethysmograph.

### EXAMPLE 14

### Exemplary Method for Determining a Stable Photoplethysmograph Blood Flow Signal Via Current Signal Stabilizers

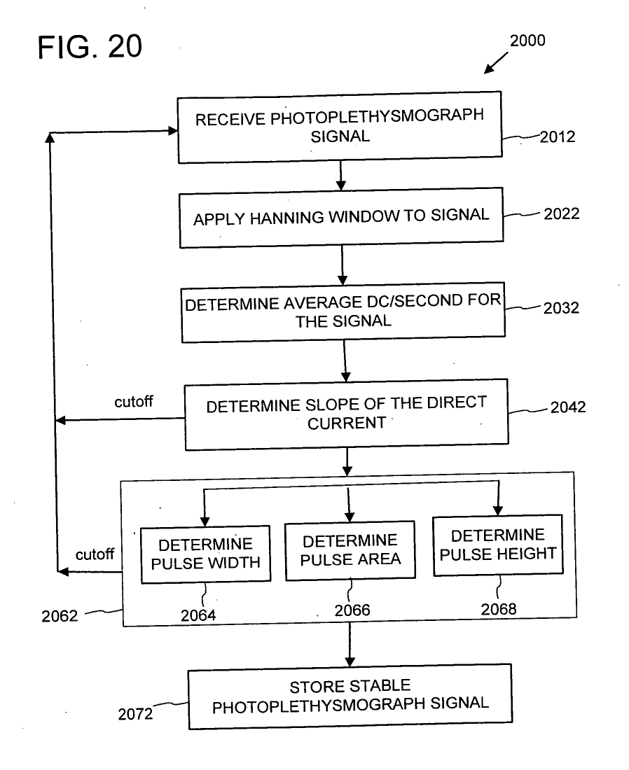

FIG. 20 shows an exemplary method for determining a stable photoplethysmograph blood flow signal of a feature via current signal stabilizers. The method can be performed, for example, by system 1900 of FIG. 19.

At 2012, a photoplethysmograph signal (e.g., the photoplethysmograph signal 1900 of FIG. 19) representing the blood flow in a feature is received.

At 2022, a hanning window is applied to the photoplethysmograph signal to remove distortion effects from spectral leakage.

At 2032, average direct current per second calculations are determined for the signal.

At 2042, the slope of the direct current signal is determined. A cutoff or predefined value can be used to determine an optimal stable photoplethysmograph blood flow signal of the feature. If the slope does not fall within the specified cutoff, the method continues at 2012 in order to determine a stable photoplethysmograph signal.

Otherwise, the method continues at 2062 with analysis of the alternating current component of the photoplethysmograph signal. At 2064, a pulse width of the alternating current is determined from a valley-to-valley time between pulses. At 2066, a pulse area of the alternating current is determined from a valley-to-valley integral between pulses. At 2068, a pulse height of the alternating current is determined from a valley-to-peak distance of pulses. Any number of statistical measurements can be used for the analysis of the alternating current component of the photoplethysmograph signal. For example, a cutoff or predefined value, such as mean, correlation, variance, and/or standard deviation calculations of the pulse width, pulse area, and/or pulse height, can be used to determine an optimal photoplethysmograph blood flow signal of a feature. If the determined analyzed alternating current component does not fall within the specified cutoff, the method continues at 2012 in order to determine a stable photoplethysmograph signal.

Otherwise, the photoplethysmograph signal that was received and analyzed via current signal stabilizers and found to be within the predefined acceptable cutoffs is stored as a stable photoplethysmograph signal for use in further analysis in evaluating a physiological condition of a subject, as indicated at 2072.

### EXAMPLE 15

### Exemplary System for Determining a Blood Flow Characteristic of a Feature

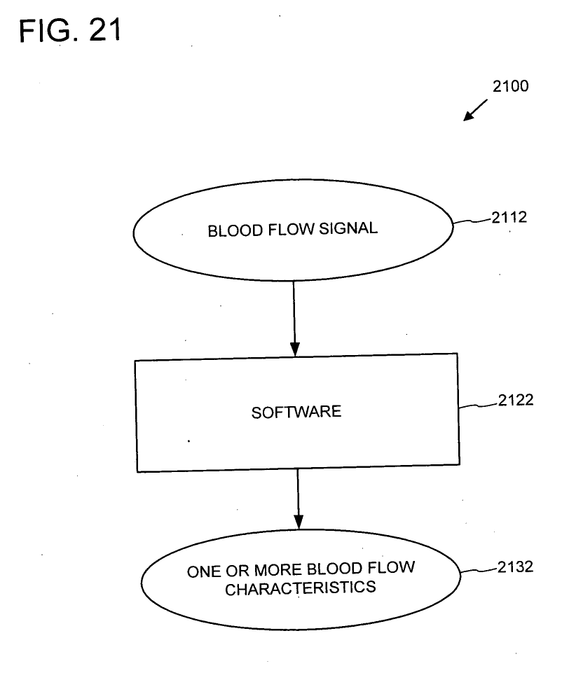

FIG. 21 shows an exemplary system 2100 for determining one or more blood flow characteristics 2132 from a blood flow signal 2112 of a feature. In particular embodiment, the blood flow signal comprises a stable blood flow signal 1732 of FIG. 17.

The blood flow signal 2112 is processed by software 2122 to determine one or more blood flow characteristics of the feature. The software 2122 can employ any combination of the technologies described herein.

In any of the examples described herein, a variety of blood flow characteristics can be determined via the blood flow signal 2112. For example, mean pulse parameter characteristics and characteristics of blood flow based on the change in blood flow can be determined via the blood flow signal.

Further, blood flow characteristics 2132 can be depicted via user interfaces. For example, a graphical depiction of the blood flow characteristics can be displayed to a human classifier, who decides what action, if any, to take. Such user interfaces can allow manipulation of the graphical depiction, such as comparison with other blood flow characteristics from the subject and/or other subjects with specified conditions.

### EXAMPLE 16

### Exemplary Method for Determining a Blood Flow Characteristic of a Feature

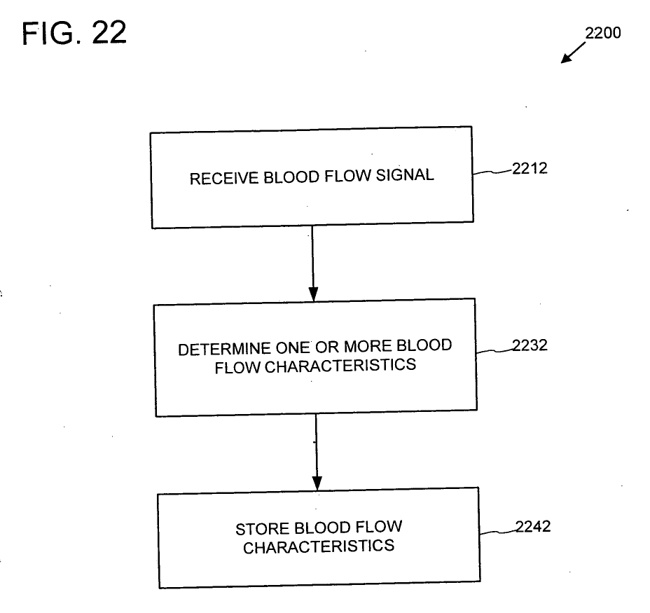

FIG. 22 shows an exemplary method 2200 for determining one or more blood flow characteristics of a feature. The method can be performed, for example, by the system 2100 of FIG. 21. The method 2200 and any of the other methods described herein can be performed by computer-executable instructions stored on one or more computer-readable media.

At 2212, a blood flow signal (e.g. the blood flow signal 2112 of FIG. 21) representing the blood flow of a feature is received.

At 2232, one or more blood flow characteristics of the feature are determined. As described in the examples below, a variety of techniques can be used for determining such a characteristic. For example, characteristics can be determined from a mean pulse signal as well as from pulse signals representing a change in blood flow.

At 2242, the blood flow characteristics can be stored in one or more computer-readable media.

### EXAMPLE 17

### Exemplary System for Evaluating a Physiological Condition of a Subject Via a Blood Flow Characteristic of a Feature

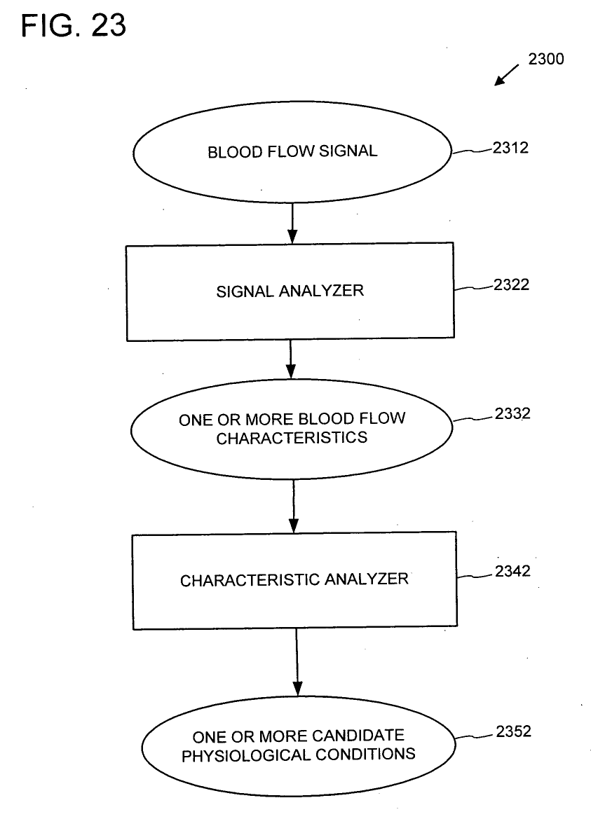

FIG. 23 shows an exemplary system 2300 for evaluating a physiological condition of a subject via one or more blood flow characteristics of a feature. A signal analyzer 2322 can receive a blood flow signal 2312 (e.g. the blood flow signal 2112 of FIG. 21) and determine one or more blood flow characteristics 2332 of the feature. A characteristic analyzer 2342 can then receive the blood flow characteristics 2332 and determine one or more candidate physiological conditions 2352 of the subject.

### EXAMPLE 18

### Exemplary Method for Evaluating a Physiological Condition of a Subject Via a Blood Flow Characteristic in a Feature

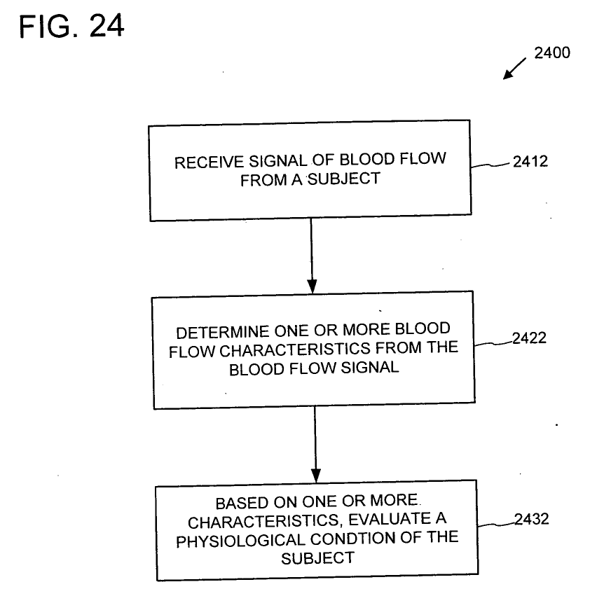

FIG. 24 shows an exemplary method 2400 for evaluating a physiological condition of a subject via one or more blood flow characteristics in a feature. The method can be performed, for example, by the system 2300 of FIG. 23. The method 2400 and any of the other methods described herein can be performed by computer-executable instructions stored on one or more computer-readable media.

At 2412, a blood flow signal from a feature (e.g. the blood flow signal 2112 of FIG. 21) is received.

At 2422, one or more blood flow characteristics of the blood flow signal are determined. As described in the examples, a variety of techniques can be used for determining such characteristics. For example, characteristics can be determined from a mean pulse signal as well as from pulse signals representing a change in blood flow.

At 2432, a physiological condition of a subject can be evaluated based on one or more blood flow characteristics.

### EXAMPLE 19

### Exemplary System for Classifying Blood Flow Characteristics of a Feature for Evaluating a Physiological Condition of a Subject

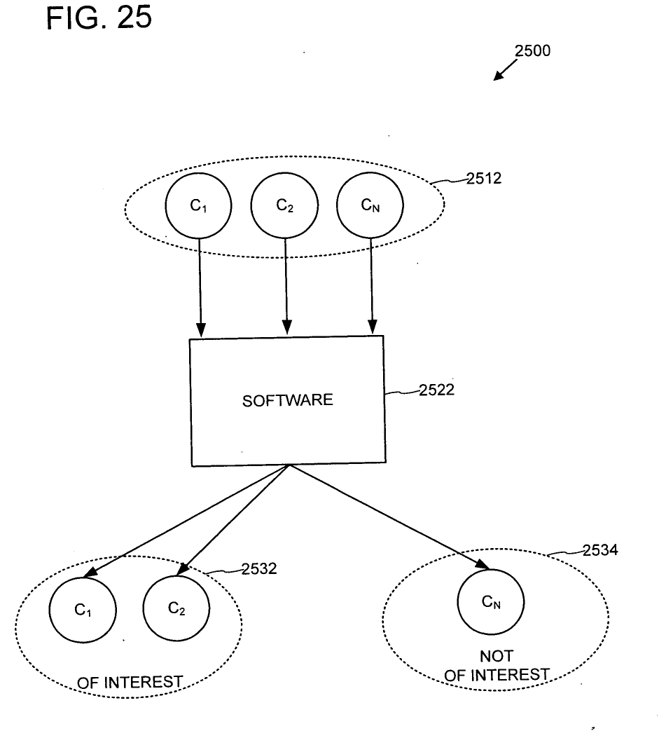

FIG. 25 shows an exemplary system 2500 for processing a plurality of blood flow characteristics of a feature with software to classify candidate blood flow characteristics of interest for evaluating a physiological condition of a subject. A plurality of blood flow characteristics of a feature 2512 (e.g. C₁, C₂, C_{N}) are received by software 2522, which classifies the blood flow characteristics as candidate blood flow characteristics of interest 2532 (e.g. C₁, C₂) or blood flow characteristics not of interest 2534 (e.g. C_{N}). For example, in a system for evaluating a physiological condition of a subject, such as system 2300 of FIG. 23, a blood flow characteristic can be classified as of interest (for example, the characteristic is associated with a physiological condition) or not of interest (for example, the characteristic is not associated with a physiological condition). Additional classifications are also possible (e.g. classifying a candidate blood flow characteristic as being associated with multiple physiological conditions or reclassifying characteristics based on probabilities of being associated with one or more conditions).

Software 2522 can employ any combination of the technologies described herein.

Blood flow characteristics 2512 can take a variety of forms. For example, the characteristics can be predetermined to be blood flow characteristics of interest via medical professional determination, software (not shown), or any combination thereof. They can then be processed by software 2522 to more accurately determine candidate blood flow characteristics of interest.

The classifications 2532 and 2534 can be represented in a variety of ways. For example, a blood flow characteristic can be explicitly labeled as being of interest or not of interest. Alternatively, a list of blood flow characteristics can be maintained, and blood flow characteristics determined not to be of interest can simply be removed from the list. In some cases, a blood flow characteristic need not be explicitly classified. For example, processing may fail to find a blood flow characteristic of interest because a subject does not have any blood flow characteristics that are associated with a physiological condition that is being evaluated. In such a case, the blood flow characteristics can simply be omitted from further presentation.

The action of classification can be added to any of the methods described herein in which blood flow characteristics are determined.

### EXAMPLE 20

### Exemplary Blood Flow Characteristics Based on Blood Flow Signal

In any of the examples herein, a variety of blood flow characteristics can be computed based on a blood flow signal. For example, blood flow characteristics can be determined from a mean pulse signal as well as from pulse signals representing a change in blood flow.

Such characteristics determined from a mean pulse signal can include, without limitation, pulse parameters such as minimum rise time (MRT), stiffness index (SI), frequency analysis of harmonics (FFT), and normalized pulse shape analysis. Determination of MRT is described in Gavish B., 1987, “Photoplethysmographic characterization of the vascular wall by a new parameter-minimum rise-time: age dependence on health,” Microcirc. Endoth. Lymphatics 3, pages 281-96. Determination of SI is described in Millasseau S. C., Kelly R. P., Ritter J. M. and Chowienczyk P. J., 2002 “Determination of age-related increases in large artery stiffness by digital pulse contour analysis,” Clinical Science 103, pages 371-77. Determination of FFT is described in Sherebrin M. H. and Sherebrin R. Z., 1990, “Frequency analysis of the peripheral pulse wave detected in the finger with a photoplethysmograph,” IEEE Trans. on Biomed. Eng. 37, pages 313-17. Determination of normalized pulse shape is described in Oliva I., Ipser J., Roztocil K., and Guttenbergerova K., 1976, “Fourier analysis of the pulse wave in obliterating arteriosclerosis,” VASA 5, pages 95-100; and Allen J. and Murray A., 2003, “Age-related changes in the characteristics of the photoplethysmographic pulse shape at various body sites,” Physiol Meas. 24, pages 297-307.

Such characteristics determined from pulse signals representing a change in blood flow can include, without limitation, a rate of return of blood flow into a feature, a difference between blood flow before a force is applied to the feature and blood flow after reducing or removing the force (including differences in characteristics of a photoplethysmograph blood flow signal, such as the direct and alternating current components, and pulse volume), and a time interval representative of blood volume return. Statistical measurements such as means, standard deviations, normalization, double normalization and the like can be used to further describe characteristics.

### EXAMPLE 21

### Exemplary Classification of Blood Flow Characteristics of Features Based on Blood Flow Signal

The blood flow characteristics computed for a feature of a subject can be compared with paradigmatic blood flow characteristics of features of subjects with known physiological conditions. Based on determining that the feature of the subject has blood flow characteristics associated with a physiological condition, the blood flow characteristics can be classified accordingly.

To achieve classification, blood flow characteristics from the subject and subjects with know physiological conditions can be used as input to a classifier, such as a rule-based system, a neural network, or a support vector machine. The classifier can draw upon the various characteristics to classify blood flow characteristics as candidate blood flow characteristics of interest and/or blood flow characteristics not of interest. For example, the blood flow characteristic can be removed from a list of blood flow characteristics or depicted distinctly in a visual depiction.

### EXAMPLE 22

### Exemplary Physiological Conditions

A physiological condition can include any condition that demonstrates critical changes in peripheral circulation, including heart disease, peripheral vascular disease, diabetes, Raynaud's phenomenon, hand-arm vibration syndrome (HAVS), or the like. Additionally, the technologies described herein can be applied to evaluate physiological conditions such as age and handedness.

### EXAMPLE 23

### Exemplary System for Determining a Blood Flow Characteristic of a Feature Via Mean Pulse Analysis

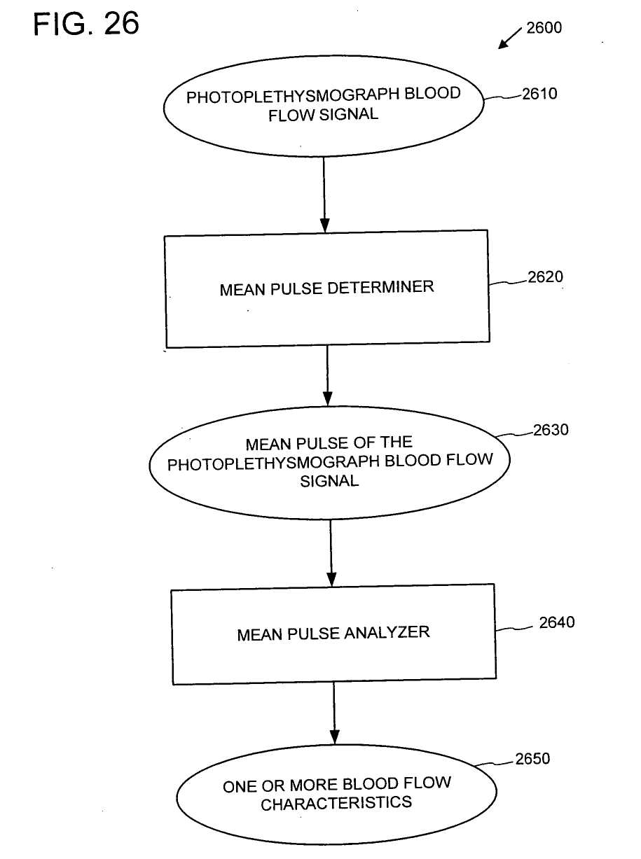

FIG. 26 shows an exemplary system 2600 for determining a blood flow characteristic of a feature via mean pulse analysis. A mean pulse determiner 2620 can receive a photoplethysmograph blood flow signal 2610 (for example, the blood flow signal 2112 of FIG. 21 or the stable photoplethysmograph blood flow signal 1962 of FIG. 19) and determine the mean pulse of the photoplethysmograph blood flow signal. A mean pulse analyzer 2640 (for example, the signal analyzer 2322 of FIG. 23) can then receive the mean pulse of the photoplethysmograph blood flow signal and determine one or more blood flow characteristics 2650 (for example, the one or more blood flow characteristics 2132 of FIG. 21).

### EXAMPLE 24

### Exemplary Method for Determining a Blood Flow Characteristic of a Feature Via Mean Pulse Analysis

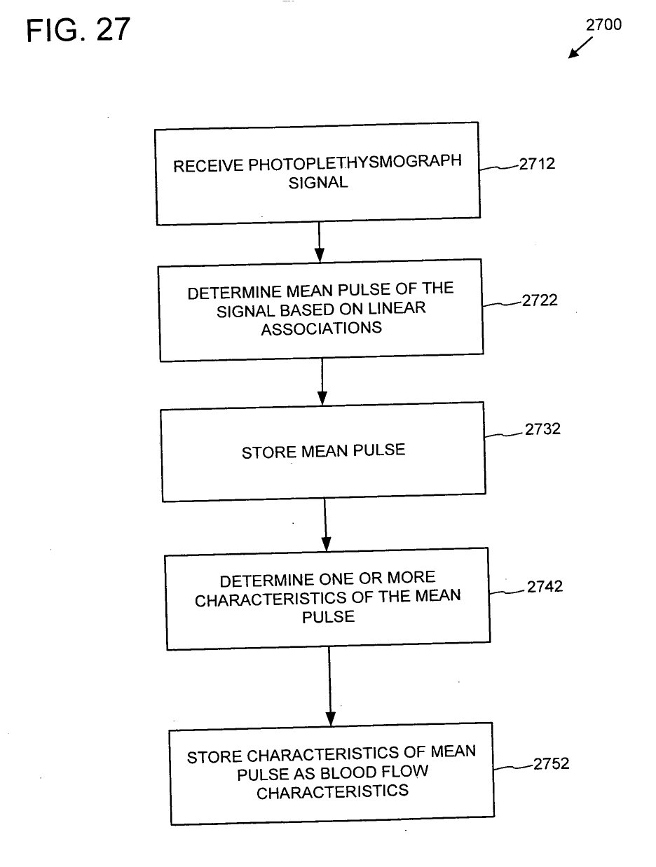

FIG. 27 shows an exemplary method for determining a blood flow characteristic of a feature via mean pulse analysis. The method 2700 can be performed, for example, by the system 2600 of FIG. 26.

At 2712, a photoplethysmograph blood flow signal (for example, blood flow signal 2112 of FIG. 21 or stable photoplethysmograph blood flow signal 1962 of FIG. 19) representing the blood flow of a feature is received.

At 2722, the mean pulse of the photoplethysmograph signal is determined based on linear associations between pulses within the photoplethysmograph signal.

At 2732, the mean pulse of the photoplethysmograph signal is stored.

At 2742, a characteristic of the mean pulse is determined.

At 2752, a characteristic of the mean pulse is stored as a blood flow characteristic of a feature.

### EXAMPLE 25

### Exemplary Method for Determining a Blood Flow Characteristic of a Feature Via Mean Pulse Analysis

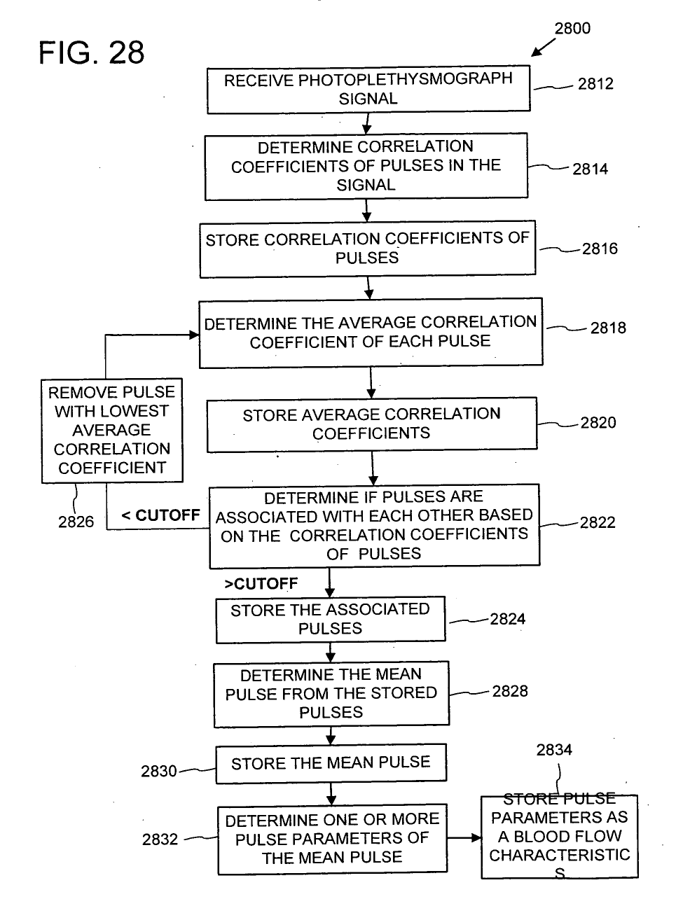

FIG. 28 shows another exemplary method for determining a blood flow characteristic of a feature via mean pulse analysis. The method 2800 can be performed, for example, by the system 2600 of FIG. 26.

At 2812, a photoplethysmograph blood flow signal (for example, the blood flow signal 2112 of FIG. 21 or the stable photoplethysmograph blood flow signal 1962 of FIG. 19) representing the blood flow of a feature is received.

At 2814, correlation coefficients (or “correlations”) of pulses in the photoplethysmograph signal are determined. One method that can be used is determining a matrix of correlations between pulses as shown in equation (1), where R=the matrix of correlations between pulses, m=pulses, r=the correlation between two pulses, and xₗ and xₖ represent each pair of pulses, sₗ and sₖ are the sample standard deviations the pulses. Thus, each row of the matrix will contain values that represent correlation coefficients of one of the pulses with respect to the other pulses in the signal. For example, in the first row, r₁₁ represents the correlation between pulse 1 with itself, r₁₂ represents the correlation between pulse 1 and pulse 2, and so on; and, in the second row, r₂₁ represents the correlation between pulse 2 and pulse 1, r₂₂ represents the correlation between pulse 2 with itself, and so on.

$$R = ( \begin{matrix}{r}_{11} & ⋯ & {r}_{1 m} \\ ⋮ & ⋰ & ⋮ \\ {r}_{m 1} & ⋯ & {r}_{m m}\end{matrix} ) where {r}_{lk} = {\sum }_{i = 1}^{n} \frac{( {x}_{li} - {\overline{x}}_{l} ) ( {x}_{ki} - {\overline{x}}_{k} )}{{s}_{l} {s}_{k} ( n - 1 )} \qquad ( 1 )$$

At 2818, the average correlation coefficient of each pulse is determined. One method that can be used is to average each row of matrix R in equation (1).

At 2820, the average correlation coefficient of each pulse is stored.

At 2822, a linear association between pulses can be determined based on the correlation coefficients of pulses. A predefined cutoff or threshold can be used to determine optimal association between pulses. For example, a cutoff of 0.95 can be used. If any pulse's average correlation coefficient is determined to be less than the specified cutoff, then the pulse with the smallest average correlation coefficient is removed at 2826 and the method continues at 2818. An additional cutoff utilizing the correlation coefficients that make up the average correlation coefficient for each pulse can be used to further improve accuracy. For example, if any pulse's association (correlation coefficient) with any other pulse is determined to be less than the specified cutoff (for example, if a pulse's average correlation coefficient is equal to or above the cutoff, but a correlation coefficient that makes up that average is below the cutoff), then the pulse with the smallest average correlation coefficient is removed at 2826 and the method continues at 2818. Utilizing the additional cutoff results in a matrix in which all correlation coefficients are at or above the specified cutoff.

Otherwise, the method continues at 2824 where the associated pulses are stored.

At 2828, a mean pulse of the stored associated pulses is determined. One method is to average the stored associated pulses.

At 2830, the mean pulse is stored.

At 2832, one or more pulse parameters of the mean pulse are determined. Pulse parameters can include minimum rise time, stiffness index, frequency analysis of harmonics, and normalized pulse shape analysis.

At 2834, the pulse parameters of the mean pulse are stored as a blood flow characteristic of the feature.

### EXAMPLE 26

### Exemplary Method for Determining a Blood Flow Characteristic of a Feature Via Mean Pulse Analysis

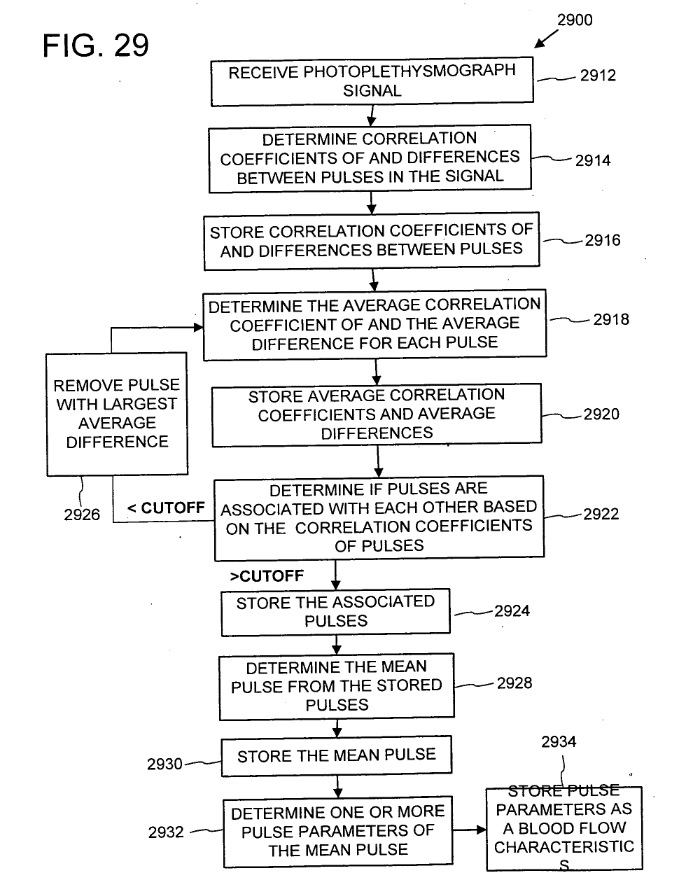

FIG. 29 shows yet another exemplary method for determining a blood flow characteristic of a feature via mean pulse analysis. The method 2900 can be performed, for example, by the system 2600 of FIG. 26.

At 2912, a photoplethysmograph blood flow signal (for example, the blood flow signal 2112 of FIG. 21 or the stable photoplethysmograph blood flow signal 1962 of FIG. 19) representing the blood flow of a feature is received.

At 2914, correlation coefficients (or “correlations”) of pulses and differences between pulses in the photoplethysmograph signal are determined. One method that can be used to determine correlation coefficients of pulses includes determining a matrix of correlations between pulses as shown in equation (1). One method that can be used to determine differences between pulses includes determining a matrix of differences between pulses as shown in equation (2), where D=the matrix of differences between pulses, m=pulses, d=the difference between two pulses, and xₗ and xₖ represent each pair of pulses, sₗ and sₖ are the sample standard deviations the pulses.

$$D = ( \begin{matrix}{d}_{11} & ⋯ & {d}_{1 m} \\ ⋮ & ⋰ & ⋮ \\ {d}_{m 1} & ⋯ & {d}_{m m}\end{matrix} ) where {d}_{lk} = {\sum }_{i = 1}^{n} \frac{{( ( {x}_{li} - {\overline{x}}_{l} ) ( {x}_{ki} - {\overline{x}}_{k} ) )}^{2}}{n} \qquad ( 2 )$$

At 2916, the correlation coefficients of pulses and the differences between pulses are stored.

At 2918, the average correlation coefficient of each pulse and the average difference for each pulse are determined. One method that can be used includes averaging each row of matrix R in equation (1) to determine the average of the correlation coefficients r for a pulse with m pulses, and averaging each row of matrix D in equation (2) to determine the average of the squared differences for a given pulse with m pulses.

At 2920, the average correlation coefficient and average of the squared difference of each pulse is stored.

At 2922, a linear association between pulses can be determined based on the correlation coefficients of pulses. A predefined cutoff or threshold can be used to determine optimal association between pulses. For example, a cutoff of 0.95 can be used. If any pulse's average correlation coefficient is determined to be less than a specified cutoff, then the pulse with the largest average difference is removed at 2926 and the method continues at 2918. An additional cutoff utilizing the correlation coefficients that make up the average correlation coefficient for each pulse can be used to further improve accuracy. For example, if any pulse's association (correlation coefficient) with any other pulse is determined to be less than the specified cutoff (for example, if a pulse's average association is equal to or greater than the cutoff, but a correlation coefficient that makes up that average is below the cutoff), then the pulse with the smallest average correlation coefficient is removed at 2826 and the method continues at 2818. Utilizing the additional cutoff results in a matrix in which all correlation coefficients are at or above the specified cutoff.

Otherwise, the method continues at 2924 where the associated pulses are stored.

At 2928, a mean pulse of the stored associated pulses is determined. One method is to average the stored associated pulses.

At 2930, the mean pulse is stored.

At 2932, one or more pulse parameters of the mean pulse are determined. Pulse parameters can include minimum rise time, stiffness index, frequency analysis of harmonics, and normalized pulse shape analysis.

At 2934, the pulse parameters of the mean pulse are stored as a blood flow characteristic of the feature.

### EXAMPLE 27

### Exemplary Depiction of a Method for Determining a Mean Pulse from a Photoplethysmograph Signal of Blood Flow in a Feature

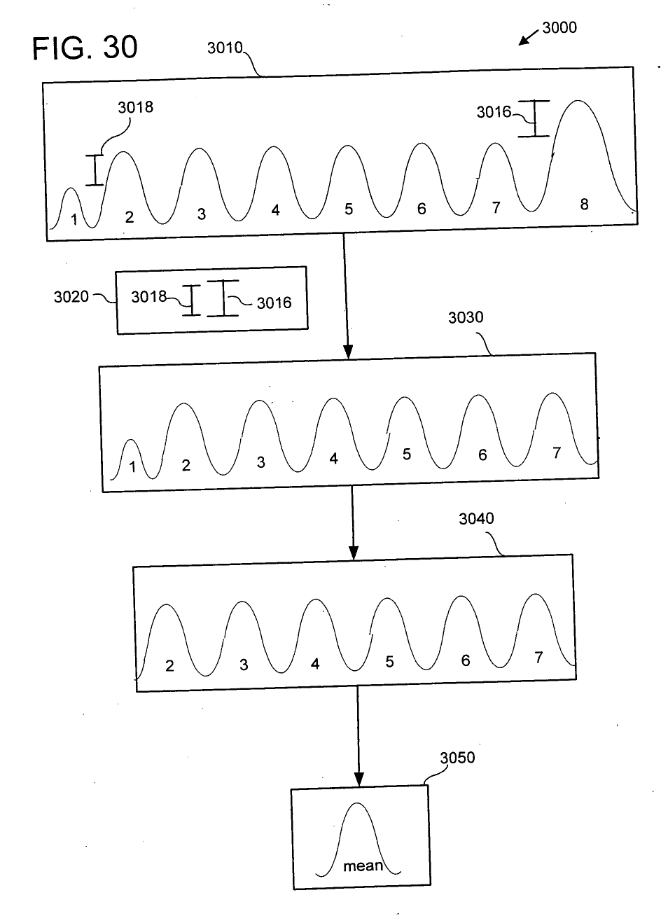

FIG. 30 shows an exemplary depiction of a method 3000 for determining a mean pulse from a photoplethysmograph blood flow signal of blood flow in a feature as described in method 2900 in FIG. 29. The method 3000 can be performed, for example, by the mean pulse determiner 2620 of system 2600 of FIG. 26.

At 3010, a photoplethysmograph blood flow signal (for example, the blood flow signal 2112 of FIG. 21 or the stable photoplethysmograph blood flow signal 1962 of FIG. 19) representing the blood flow of a feature is depicted. Pulses within the signal are labeled 1-8, with pulses 1 and 8 being visually different from the other pulses for the purpose of illustrating the method. Correlation coefficients of the pulses and differences between the pulses in the photoplethysmograph signal can be determined and analyzed to determine associated pulses. In the illustrated depiction, it was determined that not all of the pulses are associated within one another according to a threshold for the correlation coefficients of pulses. The average differences between pulse 1 and the rest of the pulses in the signal is depicted by measurement 3018, and the average difference between pulse 8 and the rest of the pulses in the signal is depicted by measurement 3016.

At 3020, the method for determining which pulse should be removed from the signal is depicted. In this example, pulse 8 with the largest average difference 3016 is removed from the signal. In other methods, the pulse with the smallest average correlation coefficient can be removed instead (for example, the removal step 2826 of method 2800 of FIG. 28).

At 3030, a modified depiction of photoplethysmograph blood flow signal 3010 with pulse 8 removed is illustrated. The average correlation coefficient of each pulse and the average difference for each pulse are re-determined for the modified signal and the association between the pulses is re-determined. In the illustrated example, it is again determined that not all of the pulses are associated with one another within the established threshold. Pulse 1 is identified as having the largest average difference and subsequently is removed from the signal.

At 3040, a modified depiction of photoplethysmograph blood flow signal 3030 is illustrated showing pulse 1 (and pulse 8) removed. The average correlation coefficient of each pulse and the average difference for each pulse are re-determined for the modified signal and the association between the pulses is pulses re-determined. In the illustrated example, it is now determined that all of the pulses that remain in the signal are associated with one another within the established threshold and a mean pulse can be determined from the pulses that remain.

At 3050, a depiction of the determined mean pulse from associated pulses 2-7 of the signal is illustrated.

### Example 28

### Exemplary Depiction of a Mean Pulse Parameter

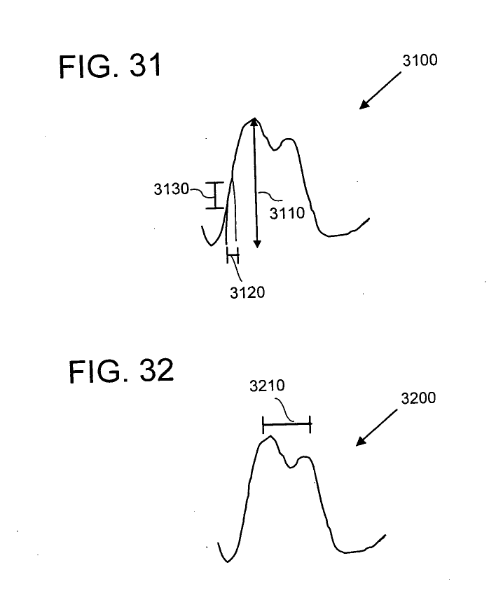

FIG. 31 shows an exemplary depiction of components of a minimum rise time pulse parameter determined from a mean pulse 3100 (for example, mean pulse 2630 of FIG. 26). Minimum rise time is a systolic parameter which can correlate with age and vascular health. Mean pulse 3100 includes a pulse height 3110, a sampling time (dt) indicated at 3120, and a maximum vertical differential (systolic portion) (Dy) indicated at 3130. Minimum rise time can be determined by equation (3):
Minimum Rise Time=Pulse height*dt/Dy (3)

### EXAMPLE 29

### Exemplary Depiction of a Mean Pulse Parameter

FIG. 32 shows an exemplary depiction of a component of a stiffness index pulse parameter determined from a mean pulse 3200 (for example, mean pulse 2630 of FIG. 26). Stiffness index is a pulse parameter than can correlate with age and vascular health. Stiffness index can be determined by equation (4), wherein ΔT=Time between systolic peak and diastolic peak of the pulse. Mean pulse 3200 includes a ΔT indicated at 3210.
Stiffness Index=Person's height/ΔT (4)

### EXAMPLE 30

### Exemplary System for Determining a Blood Flow Characteristic of a Feature Via Applying a Force to a Feature

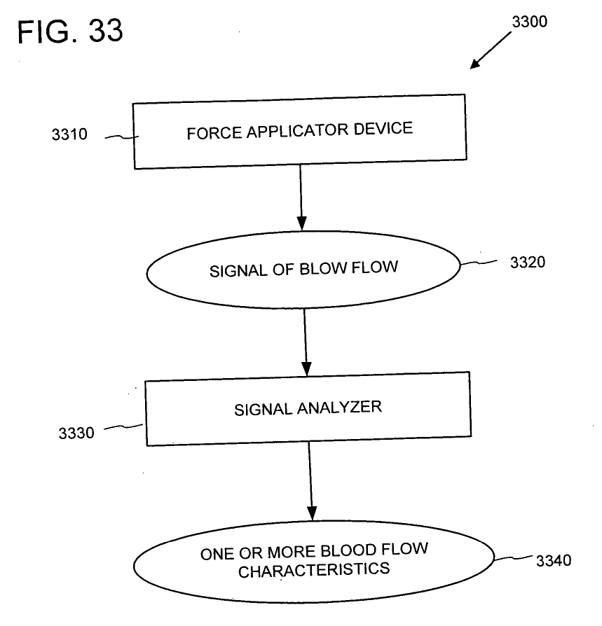

FIG. 33 shows an exemplary system 3300 for determining one or more blood flow characteristics of a feature via applying a force to a feature so as to cause a change in blood flow of the feature. A force applicator device 3310 (for example, apparatus 100 of FIG. 1, apparatus 200 of FIG. 2, apparatus 300 of FIG. 3, apparatus 500 of FIG. 5, or apparatus 700 of FIG. 7) can be used to apply a force to a feature and determine a signal of blood flow 3320 in the feature (for example, the blood flow signal 2112 of FIG. 21). A signal analyzer 3330 (for example, the signal analyzer 2322 of FIG. 23) can then receive the blood flow signal and determine one or more blood flow characteristics 3340 (for example, the one or more blood flow characteristics 2132 of FIG. 21).

### EXAMPLE 31

### Exemplary Method for Determining a Blood Flow Characteristic of a Feature Via Applying a Force to a Feature

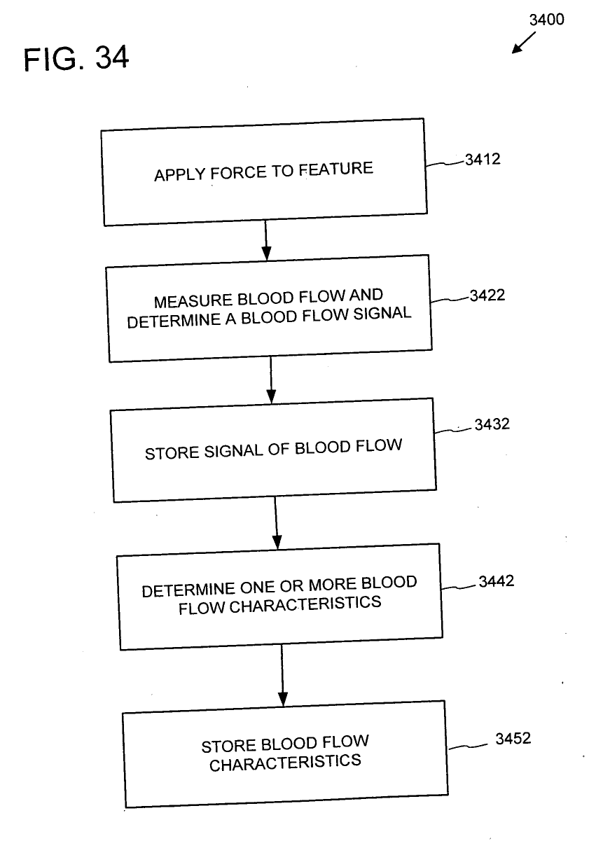

FIG. 34 shows an exemplary method for determining one or more blood flow characteristics of a feature via applying a force to a feature so as to cause a change in blood flow of the feature. The method 3400 can be performed, for example, by the system 3300 of FIG. 33.

At 3412, a force is applied to a feature. The force can be applied by a force-applying mechanism (for example, as depicted in apparatus 300 of FIG. 3, apparatus 500 of FIG. 5, and apparatus 700 of FIG. 7) or the force can also be applied by the subject (for example, as depicted in apparatus 100 of FIG. 1 and apparatus 200 of FIG. 2). A target or threshold force sufficient to prevent blood flow to the feature can be determined prior to or during application. The force can be reduced after the target or threshold force is achieved and held for a determined period of time.

At 3422, blood flow within the feature is measured and a blood flow signal is determined. For example, a blood flow detector (for example, photoplethysmograph blood flow detector 142 of FIG. 1 or camera 242 in combination with digital representation processor 242 in FIG. 2) can be used to measure the blood flow and determine a blood flow signal of the feature.

At 3432, the blood flow signal is stored.

At 3442, one or more blood flow characteristics are determined. For example, blood flow characteristics can include a rate of return of blood flow into the feature, a difference between the amount of blood flow before the force is applied and the amount of blood flow after the force is reduced, and a difference between a characteristic of a photoplethysmograph blood flow signal before the force is applied and after the force is reduced. Such characteristics of a photoplethysmograph blood flow signal can include direct and alternating current components, normalized and double normalized pulse volume, and the like.

At 3452, the blood flow characteristics are stored.

### EXAMPLE 32

### Exemplary Method for Determining a Blood Flow Characteristic of a Feature Via Applying a Force to a Feature

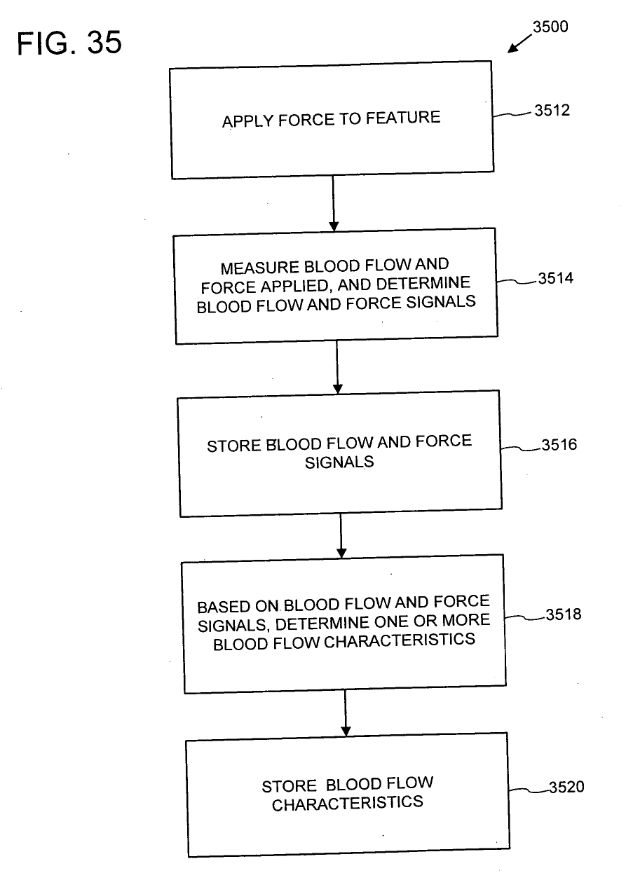

FIG. 35 shows another exemplary method for determining one or more blood flow characteristics of a feature via applying a force to a feature so as to cause a change in blood flow of the feature. The method 3500 can be performed, for example, by the system 3300 of FIG. 33.

At 3512, a force is applied to a feature. The force can be applied by a force-applying mechanism (for example, as depicted in apparatus 300 of FIG. 3, apparatus 500 of FIG. 5, and apparatus 700 of FIG. 7) or the force can also be applied by the subject (for example, as depicted in apparatus 100 of FIG. 1 and apparatus 200 of FIG. 2). A target or threshold force sufficient to prevent blood flow to the feature can be determined prior to or during application. The force can be reduced after the target or threshold force is achieved. At 3514, blood flow within the feature is measured, a blood flow signal is determined, force applied to the feature is measured, and a force signal is determined. For example, a blood flow detector (for example, photoplethysmograph blood flow detector 142 of FIG. 1 or camera 242 in combination with digital representation processor 242 in FIG. 2) can be used to measure the blood flow and determine a blood flow signal of the feature, and a force measuring device (for example, load cell force measuring device 162 of FIG. 1) can be used to measure the force applied. The measured force can be converted into a force signal. For example, measured force time series data can be converted into a smoothed force signal with spline smoothing, and then force parameters can be determined from the smoothed signal for use in blood flow analysis.

At 3516, the blood flow signal and the force signal are stored.

At 3518, one or more blood flow characteristics are determined from the blood flow and force signals. For example, a blood flow characteristic can be a time interval representative of blood volume return. Such a time interval can be the time between a time point of the measured force signal and a time point of the measured blood flow at which blood flow has returned to at least the pre-force blood flow level. In an alternative approach, the time interval can be the time between a time point of the measured force signal and a time point of the measured blood flow at which the rate of change of the blood flow after the force is released or removed is greatest. In both examples, the time point of the measured force signal can be the time point at force release or the time point at which the rate of change of the reduced force is greatest, or any other desired time point of the measured force signal, as further described below.

At 3520, the blood flow characteristics are stored

### EXAMPLE 33

### Exemplary Screen Shot Showing a Measured Force Applied to a Feature

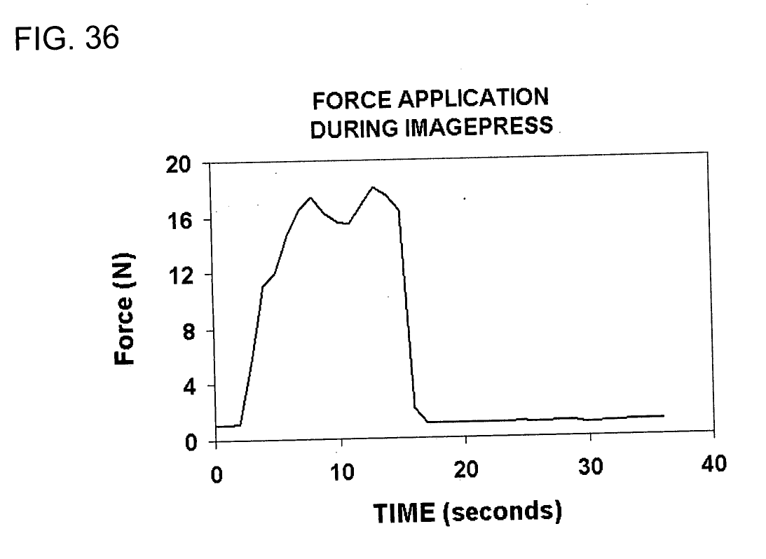

A screen shot of a view of an exemplary depiction of a measured force applied to a digit is shown in FIG. 36. The measured force can be visualized in different ways to demonstrate feature properties. FIG. 36, for example, shows a measured force represented as a smooth signal over time.

### EXAMPLE 34

### Exemplary Screen Shots Showing Changes in Blood Flow in Response to an Applied Measured Force

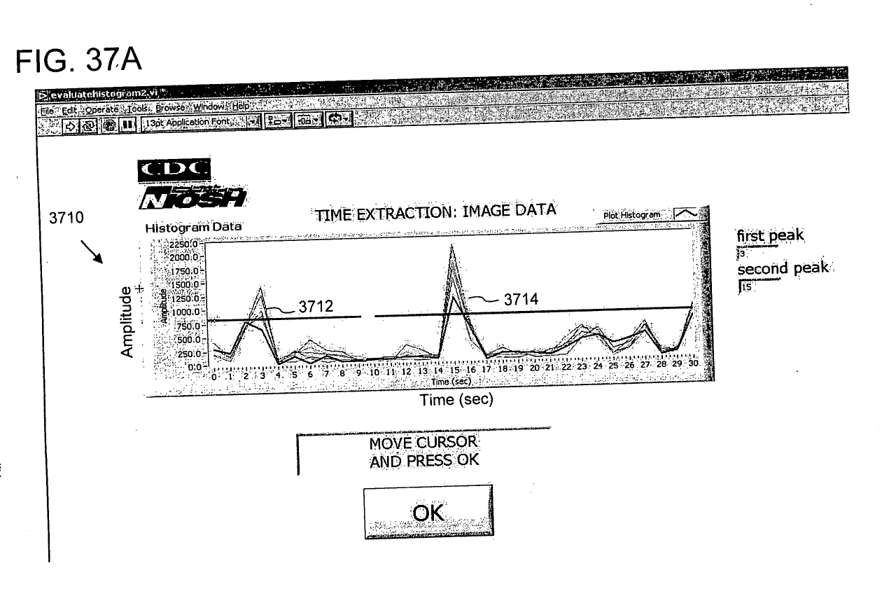

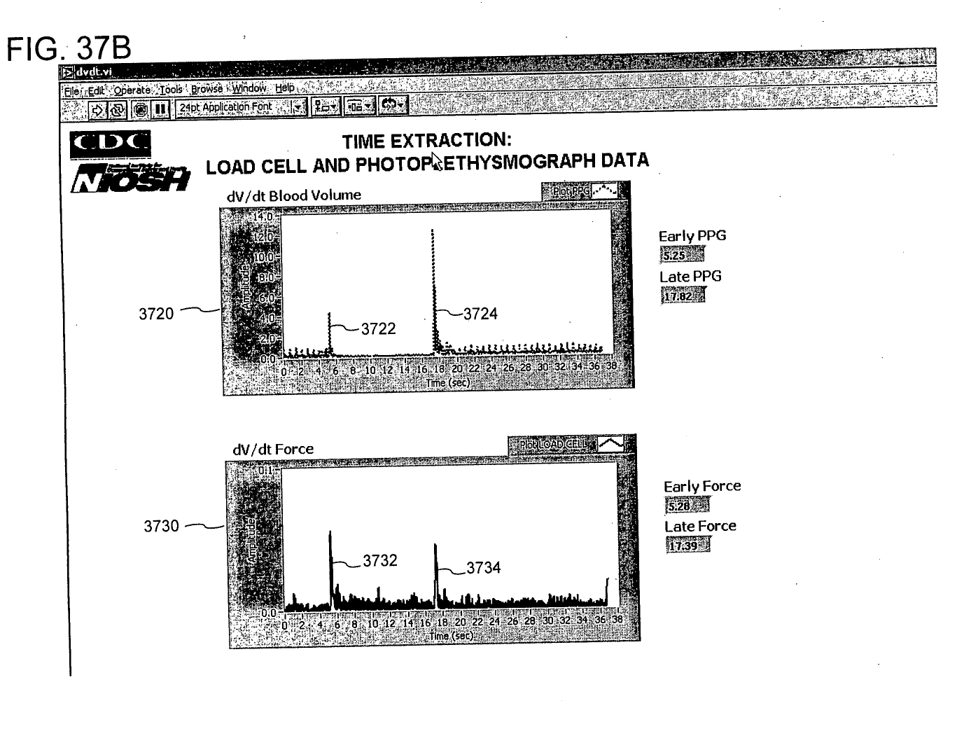

Screen shots of views of exemplary depictions of applying a varying force to a digit and the corresponding changes in blood flow in the digit are depicted in FIGS. 37A and 37B. The measured force and blood flow changes can be visualized in different ways to demonstrate feature properties.

FIG. 37A shows a visualization 3710 of the step-derivatives (slopes) of a blood flow signal derived from digital representations (for example, blood flow signal 832 of FIG. 8). Peak 3712 and peak 3714 in the signal represent the times of maximal blood flow change in response to the applied force. The first peak 3714 corresponds to when force is initially being applied to the digit to cause a reduction in blood flow in the digit, (e.g. the change in blood flow over time is greater (steeper slope)). The second peak 3714 corresponds to when the force is being reduced enough to cause an increase in blood flow in the digit (e.g. the change in blood flow over time is greater (steeper slope)).

FIG. 37B shows a visualization 3720 of the step-derivatives (slopes) of a blood flow signal derived from a photoplethysmograph detector unit (for example, blood flow signal 2112 of FIG. 21). Regular pulsations of the photoplethysmograph signal represent the baseline alternating current component of the signal, which can vary between features and subjects due to variability in composition, transparency, color, water retention, and the like. The first peak 3722 corresponds to when a force is initially being applied to the digit to cause a reduction in blood flow in the digit, (e.g. the change in blood flow over time is greater (steeper slope)). In resonse to the application of force there is a major change in the direct current component of the signal and the alternating current pulsations of the signal disappear. The second peak 3724 corresponds to when the force is being reduced enough to cause an increase in blood flow in the digit (e.g. the change in blood flow over time is greater (steeper slope)). In response to the reduction of force, there is a second major change in the direct current component of the signal and the alternating current pulsations of the signal reappear as blood volume returns to the finger. In this example, the photoplethysmograph step derivatives approach zero when blood flow is reduced to very low levels or is completely blocked. The digital representation blood signal detection method can detect small blood flow signal fluctuations in the feature during the same time period.

FIG. 37B also shows a visualization 3730 of the step-derivatives (slopes) of an applied force signal (for example, the force signal depicted in FIG. 36). The first peak 3732 corresponds to the rapid rise in applied force and the second peak 3734 corresponds to the rapid decline in applied force as it is reduced. As shown, the peak changes in the blood flow signals correspond in time with the peak changes in the force signal.

### EXAMPLE 35

### Exemplary Screen Shot Showing a Signal of Force Applied to a Feature in Combination with a Blood Flow Signal from the Feature

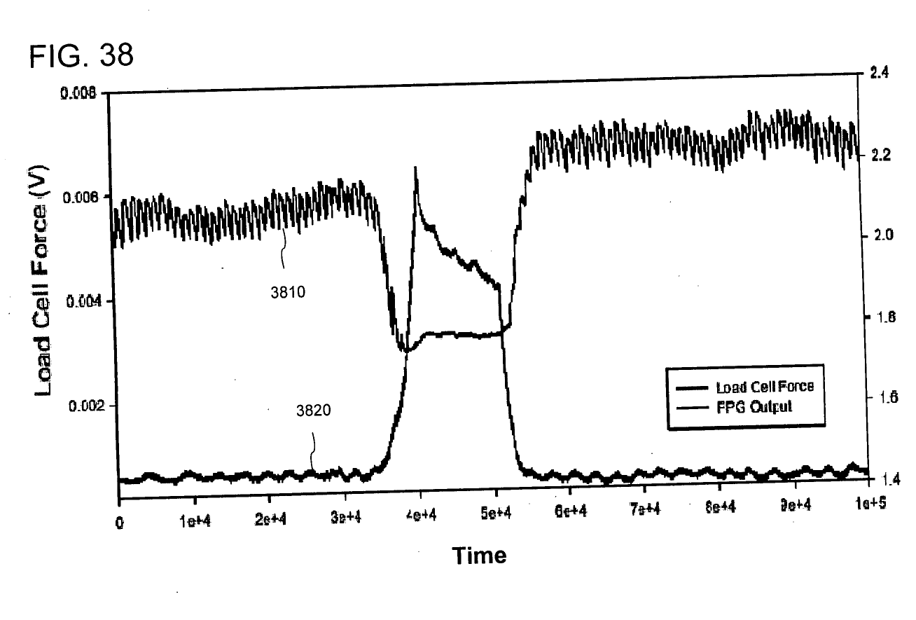

A screen shot of a view of an exemplary depiction of a measured force applied to a digit in combination with a blood flow signal from the digit is shown in FIG. 38. The measured force and blood flow signals can be visualized in different ways to demonstrate feature properties. FIG. 38 shows one visualization wherein both the force signal 3820 (for example, the force signal depicted in FIG. 36) and blood flow signal 3810 (for example, blood flow signal 2112 of FIG. 21) are illustrated on the same graph over time.

### EXAMPLE 36

### Exemplary Determination of Blood Flow Characteristics from Derived Parameters from a Force Signal of a Force Applied to a Feature and a Blood Flow Signal of a Change in Blood Flow of a Feature

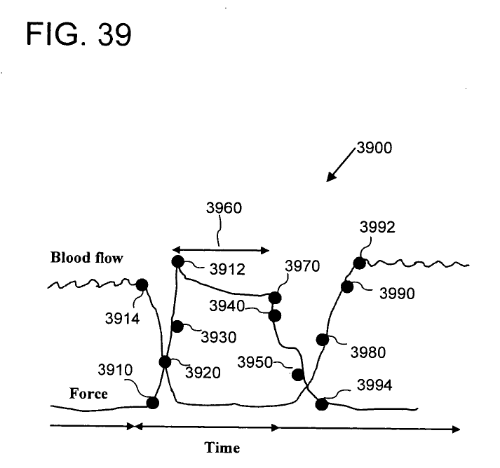

A diagram 3900 illustrating parameters of a blood flow signal of a feature and a signal of a varying force applied to the feature over a time interval is shown in FIG. 39. Various parameters (for example, time points) of the time series signals (fᵢ) can be used to determine blood flow characteristics of the feature. One method that can be used to determine useful parameters is to calculate first, second and third derivatives from smoothed signal time series data (fsᵢ) using difference equations and save them as time series as shown in equations 5, 6, and 7. The time series signals can be smoothed using spline smoothing or the like.
D¹fs=(fsᵢ−fsᵢ₋₁)/Δt (5)
D²fs=(D¹fsᵢ−D¹fsᵢ₋₁)/Δt (6)
D³fs=(D²fsᵢ−D²fsᵢ₋₁)/Δt (7)
Local maxima and minima for the time series can be calculated by locating zero crossing for D¹fs and checking the sign of D²fs.

The rising force signal can be characterized by various parameters including a peak force 3912, a peak slope 3930 located at the first zero crossing of the second derivative D²fs, a time to peak force as the difference between a time point of initiation of applying force 3910 to the time point corresponding to peak force value 3912, and an area under the curve (AUC) from the time point of initiation of applying force 3910 to the time point corresponding to the peak force value 3912. Similar corresponding parameters can be calculated for the declining blood flow signal in response to the rising force signal. For example, a time point at peak slope value 3920 of the declining blood flow can be determined.

The force plateau of the force signal can be characterized by various parameters including a plateau length 3960 as the time interval between the time point at peak force value 3912 to a time point at the end of a plateau 3970 where the force is reduced at a higher rate (the first zero crossing of the second derivative after peak force), a plateau maximum slope, and a plateau area under the curve (AUC). Similar corresponding parameters can be calculated for the trough in the blood flow signal in response to the sustained applied force.

The reduced force signal can be characterized by various parameters including a peak down slope 3940 as the time point located at the first zero crossing of the second derivative D²fs, a time interval between the time point at the end of the plateau 3970 and a peak down slope 3940, a second peak down slope 3950 as the time point located at the second zero crossing of the second derivative D²fs, a time interval between the time point at the end of the plateau 3970 and the second peak down slope 3950, and an area under the curve (AUC) of the down slope between the time point at the end of the plateau 3970 and the second peak down slope 3950. Similar corresponding parameters can be calculated for the rise in the blood flow signal in response to the reduction in the applied force. For example, a time point at peak slope value 3980 of the rising blood flow, a time point of return to at least baseline blood flow value 3990, and a time point of return to a resting blood flow value 3992 can be determined.

Blood flow characteristics such as the rate of return of blood flow and return time can be determined from parameters of a blood flow signal of a feature and a signal of force applied to the feature. For example, return time can be measured from various reference parameters including the following: (1) the time interval between the time point at the end of the plateau 3970 and the time point of return to at least baseline blood flow value 3990 or the time point of return to a resting blood flow value 3992; (2) the time interval between the time point of return of applied force to a baseline value 3994 and the time point of return to at least baseline blood flow value 3990 or the time point of return to a resting blood flow value 3992; (3) the time interval between the time point of one of the peak down slopes (3940 or 3950) and the time point of return to at least baseline blood flow value 3990 or the time point of return to a resting blood flow value 3992; (4) the time interval between the time point at the end of the plateau 3970 and the time point at peak slope value 3980 of the rising blood flow; (5) the time interval between the time point of return of applied force to a baseline value 3994 and the time point at peak slope value 3980 of the rising blood flow; and (6) the time interval between the time point of one of the peak down slopes (3940 or 3950) and the time point at peak slope value 3980 of the rising blood flow.

The difference between the resting blood flow level before a force is applied 3914 and the resting blood flow level after the force is reduced 3992 can also be determined.

### EXAMPLE 37

### Exemplary Determination of Blood Flow Characteristics from Derived Parameters from a Photoplethysmograph Blood Flow Signal of a Change in Blood Flow of a Feature

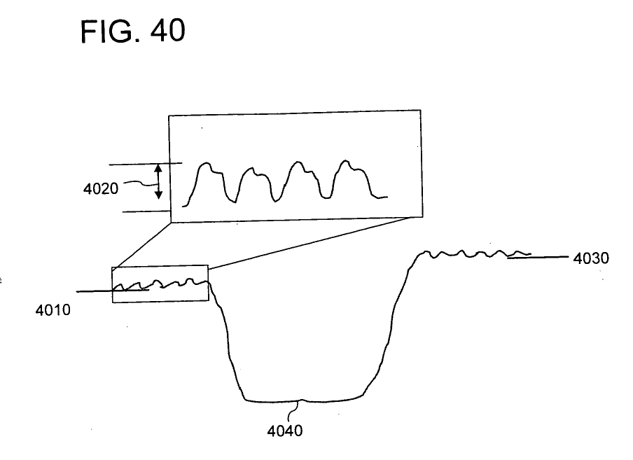

A diagram illustrating parameters (or characteristics) of a photoplethysmograph blood flow signal (for example the blood flow signal depicted in diagram 3900 of FIG. 39) showing a change in blood flow of a feature in response to an applied force to the feature over a time interval is shown in FIG. 40. Various parameters of the photoplethysmograph blood flow time series signal can be used to determine blood flow characteristics of the feature, including determining a difference between a photoplethysmograph signal parameter before the force is applied and after the force is removed or reduced. Photoplethysmograph signal parameters can include the direct current component of the signal (DC), the alternating current component of the signal (AC), the normalized pulse volume, and the double normalized pulse volume. For example, the direct current level before the force is applied is indicated at 4010 and the direct current level after the force is reduced to baseline is indicated at 4030. A blood flow characteristic can be the difference between the value of the direct current at 4010 and 4030.

Analysis of the pulse volume (PV) 4020 of the pulsatile or alternating current (AC) component of the photoplethysmograph blood flow signal can also be used to determine blood flow characteristics. Normalized pulse volume (NPV) can be determined by dividing the AC component of the signal by the baseline transmitted light level (DC) as shown in equation (8).
NPV=AC/DC (8)
DNPV=(ΔVb/Vb) (9)
NPV˜=ΔVb (10)
DNPV=NPV/ln(I/It) (11)

Double normalized pulse volume (DNPV) can be determined by dividing the pulsatile component of total blood volume ΔVb (detected by the blood flow detector) by the total amount of blood contained in both arterial and venous blood vessels Vb (detected by the blood flow detector), as shown in equation (9). Under certain conditions, the change in blood flow volume ΔVb can be approximately equal to the normalized pulse volume NPV, as shown in equation (10). Under such conditions, the double normalized pulse volume can be determined by dividing the normalized pulse volume NPV by the natural logarithm of the baseline intensity of transmitted (or reflected) light (I) from a feature (including tissue and blood) divided by the intensity of light transmitted (or reflected) (It) from only the tissue in the feature (no blood/ischemic tissue), as shown in equation (11). The value of It can be calculated as the mean trough level 4040 of the blood flow while the force is being applied, assuming no pulsations are evident (for example, the force is applied at a level equal to or higher than the threshold required to stop blood flow into the feature). The It value can also be determined by occluding blood flow in the feature and reading It as the DC value corresponding to the disappearance of pulsation.

The derivations and assumptions made when using NPV are described in Sawada Y., Tanaka G. and Yamakoshi K., 2001, “Normalized pulse volume (NPV) derived photo-plethysmographically as a more valid measure of the finger vascular tone,” Int J of Psychophysiol, 41, pages 1-10. The derivations and assumptions made when DNPV are described in Tanaka G., Sawada Y. and Yamakoshi K., 2000, “Beat-by-beat double-normalized pulse volume derived photoplethysmographically as a new quantitative index of finger vascular tone in humans,” Eur J Appl Physiol, 81, pages 148-154.

### EXAMPLE 38

### Exemplary System for Evaluating a Physiological Condition of a Subject Via a Characteristic of Blood Flow in a Feature

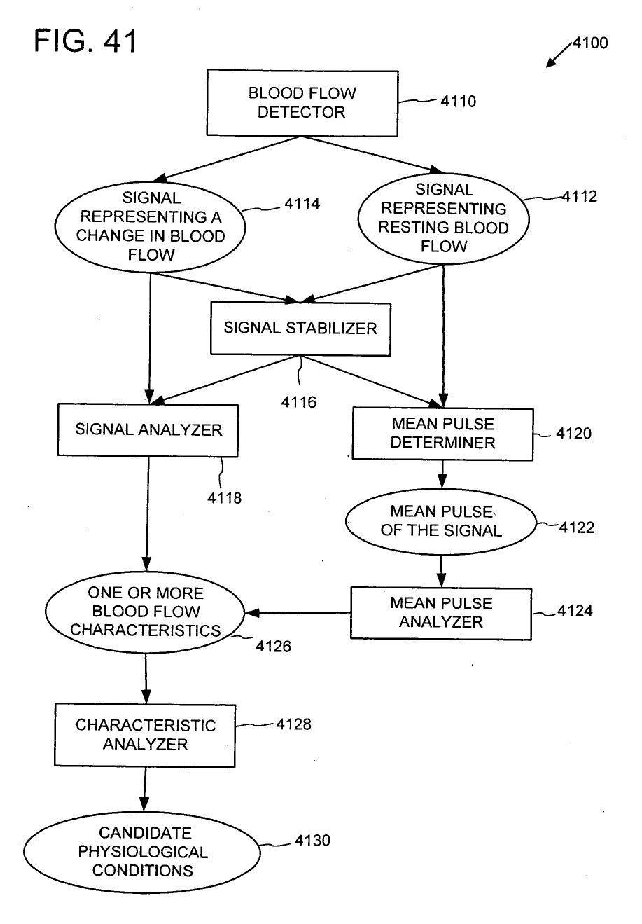

FIG. 41 shows an exemplary system 4100 for determining one or more blood flow characteristics of a feature to evaluate physiological conditions. The system 4100 can employ the technologies described herein. For example, an apparatus for evaluating blood flow (for example, apparatus 100 of FIG. 1, apparatus 200 of FIG. 2, apparatus 300 of FIG. 3, apparatus 500 of FIG. 5, or apparatus 700 of FIG. 7) can be utilized to measure a resting blood flow signal of a feature and a changing blood flow signal of the feature in response to an applied force to the feature to evaluate physiological conditions based on these signals.

As shown in the example, a signal 4112 representing a resting blood flow in a feature and a signal 4114 representing a change in blood flow in a feature in response to an applied force can be acquired from a blood flow detector 4110 (for example, system 800 of FIG. 8 or a photoplethysmograph detector can be used).

Signal 4112 can be input into a signal stabilizer 4116 (for example, system 1700 of FIG. 17 can be used for this analysis) and then the stable signal output of the signal stabilizer can be input into a mean pulse determiner 4120. Alternatively, signal 4112 can be input directly into mean pulse determiner 4120. A mean pulse of the signal 4122 can be determined and input into a mean pulse analyzer 4124, which can determine one or more blood flow characteristics 4126 (for example, system 2600 of FIG. 26 can be used for this analysis).

Signal 4114 can also be input into a signal stabilizer 4116 (for example, system 1700 of FIG. 17 can be used for this analysis), wherein the section of the signal 4114 before and after the application of force (resting blood flow sections of the overall signal 4114 representing a change in blood flow) are analyzed for stability. Subsequently, the stable signal output of the signal stabilizer 4116 can be input into a signal analyzer 4118, which can determine one or more blood flow characteristics 4126 (for example, system 3300 of FIG. 33 can be used for this analysis). Alternatively, the signal 4114 can be input directly into a signal analyzer 4118.

The one or more blood flow characteristics 4126 can be fed into a characteristic analyzer 4128, which results in one or more candidate physiological conditions 4130 (for example, system 2500 of FIG. 25 can be used for this analysis).

### EXAMPLE 39

### Exemplary Use of Results

The results of any of the technologies described herein can be presented in a variety of ways. For example, the results can be presented visually, such that candidate blood flow characteristics of interest and their associated candidate physiological conditions are shown for consideration by a human reviewer, who determines whether the physiological condition requires further investigation (for example, whether the physiological condition associated with the candidate blood flow characteristic of interest is of considerable concern to the subject's health). In this way, a number of physiological conditions which previously would not have been detected or evaluated, can be monitored and given early treatment.

### EXAMPLE 40

### Exemplary Target Force Thresholds

Target force thresholds can be defined via standardized units (e.g., Newton units). An example of determining an optimal target force threshold for restricting blood flow in a digit is described in Brumfield, A. M. and Schopper A. W., 2002, “Novel Automated Instrumentation for Finger Blood Flow Assessment,” OPTO Ireland conference poster, which is hereby incorporated by reference. An optimal target force threshold may be determined for each subject. This target force threshold may be defined to accommodate a percentile of the population or may be determined as a percentage of a subject's maximum voluntary contraction (MVC), which is a method commonly used in such measurements.

### EXAMPLE 41

### Exemplary Blood Flow Characteristic Thresholds

Appropriate thresholds can be chosen to differentiate blood flow characteristics from blood flow characteristics of interest for distinct physiological conditions. A threshold appropriate for differentiating can be determined via empirical observation or analysis of epidemiological data.

### EXAMPLE 42

### Overview of Presenting Results

Blood flow characteristics categorized as blood flow characteristics of interest can be presented in a number of ways. Additionally, blood flow characteristics classified as not of interest blood flow characteristics can be presented in a number of ways. For example, the blood flow characteristics can be presented in a digital gallery. Color coding can be used to indicate which are classified as blood flow characteristics of interest for particular physiological conditions and which are classified as not of interest.

In a fully automated system, the candidate blood flow characteristic(s) of interest for particular physiological conditions can be provided as a result. In a system with user (for example, medical professional) assistance, the blood flow characteristic(s) of interest can be presented to the user for confirmation or rejection of as being a blood flow characteristic of interest associated with a particular physiological condition. Those blood flow characteristics confirmed to be associated with physiological conditions can then be provided as a result.

### EXAMPLE 43

### Exemplary Application of Technologies

The technologies described herein can be included as part of a computer-aided detection (“CAD”) system or as a stand alone system for evaluating blood flow. By identifying characteristics of blood flow associated with physiological conditions, the technologies can increase the accuracy and effectiveness of computer-aided blood flow evaluation.

### EXAMPLE 44

### Details of Exemplary Experimental Results of Accuracy Determination of a Mean Pulse Analyzer

An automated iterative correlation mean pulse method (the determination of a mean pulse in method 2800 of FIG. 28) was implemented for 25 windows of blood flow data and tested against the mean pulse functions derived from the pulse picks of 4 independent medical professional raters. Comparisons between the automated method and each rater varied from 0.98 to 0.99 for average pulse area and 0.96 to 0.98 for average pulse width. The average correlation between the automated method-derived mean pulse and each rater's mean pulse function was greater than 0.98.

### EXAMPLE 45

### Details of Exemplary Experimental Results of Evaluation of Physiological Conditions from Resting Mean Pulse Blood Flow Analysis

Peripheral pulse measurements were collected from the right and left middle fingers of 53 subjects (median 44 years, range 31-59). Subjects were participants in an Institutional Review Board (IRB) approved study enabled through the City of Cincinnati Sewers, Water Works & Public Services, initially undertaken for the assessment of occupational vibration exposure. Eight subjects were excluded due to missing information (age or height) and/or unacceptable pulse volume data. A Raynaud's phenomenon interview was conducted to distinguish any workers who may have experienced the symptomatic color changes and/or discomfort associated with Raynaud's phenomenon or vibration white finger. This resulted in the exclusion of one female subject who presented with a borderline response. The characteristics of the resulting 43 subjects included in the analysis are presented in Table 1.

TABLE 1
Study Population

| Clinical Parameter/Age Group | 30-39 years | 40-49 years | ≧50 years | Significance |
|---|---|---|---|---|
| Subjects (male/female) | 14 (11/3) | 22 (19/3) | 9 (9/0) | NA |
| Age distribution (years) | 34 ± 2.7 | 45.3 ± 2.7 | 52.2 ± 2.8 | NA |
| Height (m) | 1.78 ± 0.11 | 1.77 ± 0.07 | 1.78 ± 0.08 | p = 0.95 |
| Systolic blood pressure (mmHg) | 130 ± 6 | 125 ± 10 | 126 ± 12 | p = 0.34 |
| Body mass index (kg/m²) | 31.8 ± 5.4 | 32.3 ± 5.7 | 31.1 ± 6.2 | p = 0.91 |
| Weight (kg) | 99.9 ± 19.1 | 99.9 ± 17.7 | 94.0 ± 11.4 | p = 0.76 |
| Heart rate (min⁻¹) | 70.9 ± 12.1 | 71.3 ± 10 | 70.7 ± 7.2 | p = 0.98 |

Subjects were asked to avoid caffeine and smoking for two hours prior to testing. The procedure was explained during acclimation to the testing environment, which was a quiet room maintained at 23±0.8° C.

Measurements were made using apparatus 100 of FIG. 1 to eliminate the need for, and remove variations due to, physical attachment of the sensor. The photoplethysmograph detector 142 comprised a Biopac transducer which was embedded into recess 124 of lever 132. Cover 152 comprised an Edmund Industrial Optics® cold mirror, which eliminated potential sensor deformation that could affect signal integrity. Peripheral pulse measurements were recorded for one minute with the subject's arm positioned at heart level and finger resting in contact with the mirror over the sensor. Approximately 60 seconds of resting data were acquired from each hand using a 16-bit DAQCard AI-16XE-50 (1024 Hz). The signal was amplified using a PPG100C photoplethysmogram amplifier module (Biopac) with a frequency response of DC to 10 Hz. A digital filter (Butterworth, highpass, 0.5 Hz) was applied to the data prior to analysis to eliminate low frequency baseline fluctuations. All processing and post-processing programs were written in LabVIEW® (National Instruments®).

The determination of a mean pulse in method 2800 of FIG. 28 was implemented to determine a mean pulse from a window of resting photoplethysmograph data. The automated iterative correlation procedure provided a mean pulse determination optimized for contour similarity, amidst movement and damping artifacts normally present in such data. The clinical validity of the method has been demonstrated previously (see Example 44), by comparing the computer-derived mean pulse to those derived independently by each member of a “gold standard” panel (two clinicians and two physiologists). Additionally, normalization of the mean pulse function was performed for overall shape assessment and to eliminate variability due to heart rate differences. This was achieved by normalizing the amplitude and dividing the width of the pulse into 100 equal divisions of time. The normalized mean pulses within each age group were averaged to yield a group pulse shape.

The minimum rise time MRT (see Example 28) was determined automatically from the derived mean pulse function of each subject. The high sampling rate necessitated the resampling of the data at a lower rate following the application of a moving average filter (window of 11) in order to accurately determine a maximal vertical differential (systolic portion) Dy. Care was taken such that the integrity of the pulse contour was maintained.

The digital pulse wave characteristically exhibits a systolic peak as a result of the direct pressure wave from the left ventricle; and a diastolic peak or inflection from reflections of the pressure wave by arteries of the lower body. The time between these two peaks (ΔT) is a coarse measure of transit time between the subclavian artery and such reflection sites and has been used to define a noninvasive measure of large artery stiffness. ΔT and Stiffness Index SI (see Example 29) were determined automatically from the derived mean pulse function of each subject.

The SI, MRT, and ΔT data for the right and left hand were shown to be significantly correlated as presented in Table 2.

TABLE 2
Paired Samples Correlations

| Parameter | Correlation | Significance |
|---|---|---|
| MRT Right & MRT Left | 0.500 | p = 0.002 |
| SI Right & SI Left | 0.779 | p < 0.001 |
| ΔT Right & ΔT Left | 0.781 | P < 0.001 |
| MRT Right & SI Right | 0.689 | p < 0.001 |
| MRT Right & ΔT Right | −0.667 | p < 0.001 |
| MRT Left & SI Left | 0.723 | p < 0.001 |
| MRT Left & ΔT Left | −0.693 | p < 0.001 |

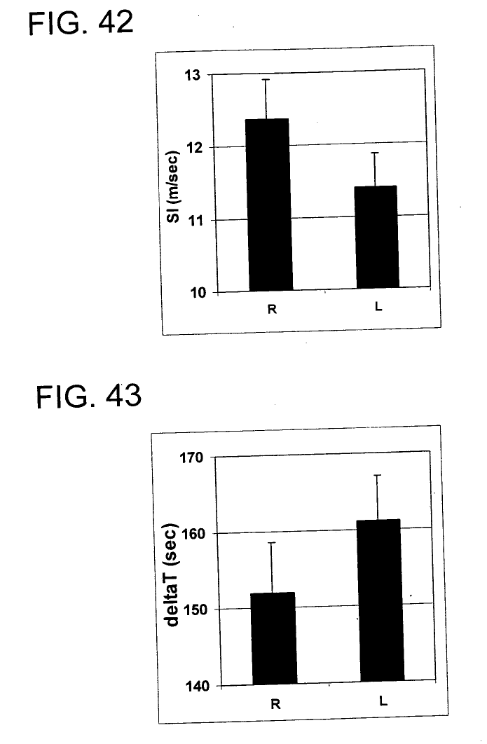

For each hand, the MRT calculations also strongly correlated with corresponding SI and ΔT values. FIG. 42 shows results of the mean stiffness index determinations of the right and left hands of the population. The right hand mean stiffness index was significantly higher (p=0.009) than the left hand mean stiffness index. FIG. 43 shows results of the related mean ΔT determinations of the right and left hands of the population. The right hand mean ΔT was significantly lower (p=0.034) than the left hand mean ΔT.

TABLE 3
Age correlations for right and left hand measurements

AGE

| PARAMETER | HAND | R | P value |
|---|---|---|---|
| Minimum Rise Time (sec) | R | 0.631 | <0.001 |
| | L | 0.347 | 0.033 |
| Stiffness Index (m/sec) | R | 0.546 | 0.001 |
| | L | 0.433 | 0.012 |
| Δ T(sec) | R | −0.495 | 0.002 |
| | L | −0.315 | 0.054 |

All parameters, MRT, SI, and ΔT, were found to significantly correlate with age (Table 3). The ANOVA results more distinctly illustrate the significance of age, with p values<0.01 for right hand measurements of all three parameters (MRT p<0.001, SI p=0.002, and ΔT p=0.009). The left hand measurement of SI was also significant with age in the ANOVA (p=0.026). Post-hoc analyses on the right hand measurements revealed significant differences between three age groups (Age Group I=30-39 years, Age Group II=40-49 years, and Age Group III=≧50). Groups I and II revealed significant differences for the MRT (p<0.001) and SI (p=0.022) parameters and a marginally a significant difference for the ΔT (p=0.073) parameter. Groups I and III revealed significant differences for the MRT (p<0.001), SI (p=0.012), and ΔT (p=0.010) parameters.

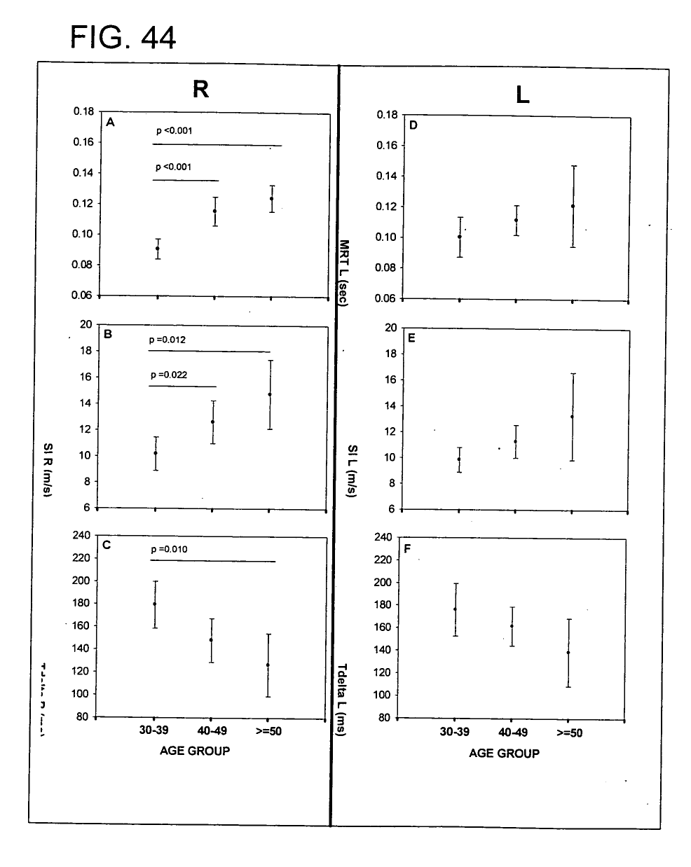

Characteristics of the mean pulse for the left and right hand measurements for each age group are shown in FIG. 44, wherein the error bars represent 95% confidence intervals. All three parameters determined from the mean pulse measurement of the right hand of the 30-39 age group can be significantly differentiated from those of the older age groups (40-49 and >50), indicating increasing MRT and SI (and the related decrease in ΔT) with age.

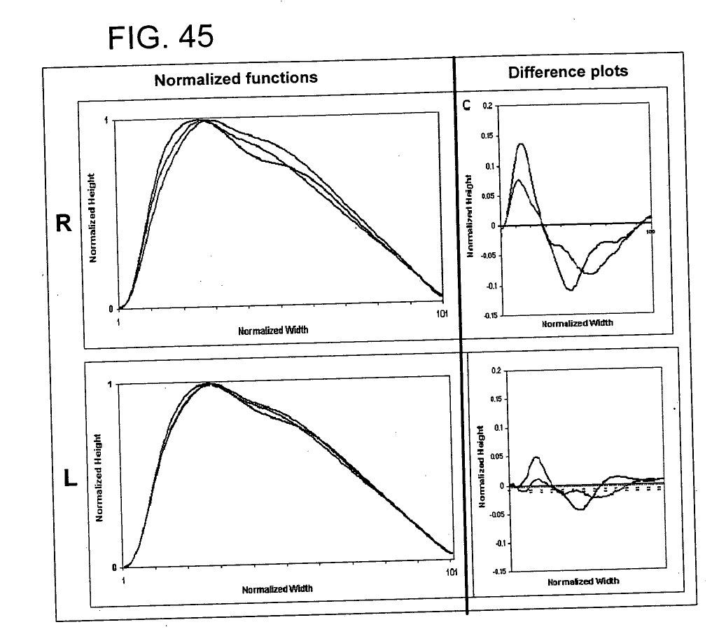

Normalized mean pulse functions and difference plots for the left (L) and right (R) hands for the age groups are depicted in FIG. 45. The normalized functions provide an illustration of the pulse shapes between groups. The mean group pulse of the oldest group was subtracted from those of the 30-39 and 40-49 age groups to determine the difference plots. Consistent with the parameters under study, the group pulse contours confirm that changes with age predominantly reside in the systolic slope and the dicrotic notch occurrence, as evidenced in the difference plots. The magnitudes of such shape changes are clearly more distinctive in the right hand. To further demonstrate these distinctions, two parameters used to derive the minimum rise time and the stiffness index have been calculated from the normalized mean pulse function. The maximum vertical differential (dY) and ΔT values were calculated from the normalized mean pulses within each age group. Findings were similar to the related parameters derived from non-normalized data. ANOVA p values were significant for dY (p<0.001) and ΔT (p=0.006) values of the right hand. Post-hoc analyses demonstrated that dY was significantly different between Groups I and II (p=0.046), Groups I and III (p=0.003), and marginally significant between Groups II and III (p=0.054). AT was shown to be significantly different between Groups I and III (p=0.020).

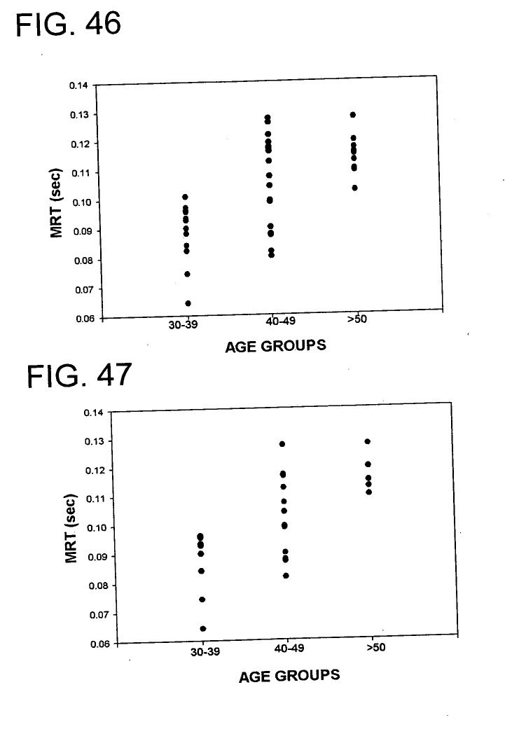

It should be mentioned that while subjects were not eliminated due to smoking, high blood pressure, diabetes, or other confounders, analyses were performed that demonstrated that such consideration indicated little or no effect on the overall results. FIG. 46 shows the subject population MRT results of all subjects and FIG. 47 shows the population following elimination of subjects who smoke, have high blood pressure, and/or are diabetic.

Initial calculations of MRT, SI, and ΔT, were performed on data which had not been manipulated beyond the instrumentation's 20 Hz lowpass filter. This conservative approach was taken given the variations between individuals, and in an effort to maintain the integrity of the original signal, particularly with regard to shape. In order to process the data in a manner consistent with previous protocols, a highpass digital filter (Butterworth, 0.5 Hz) was employed prior to the implementation of the automated algorithm. While these are the results herein and while this did eliminate some low frequency baseline fluctuations, it is noteworthy that the overall results of the study remained unchanged.

### EXAMPLE 46

### Details of Exemplary Experimental Results of Evaluation of Physiological Conditions from Analysis of an Induced Change in Blood Flow of a Feature

In this example, apparatus 200 shown in FIG. 2 was utilized to collect blood flow data from multiple subjects for the purpose of evaluating physiological conditions of the subjects. A LabVIEW® interface, enabling simultaneous image and data acquisition, provides a basis for the standardization of the force application and duration. The raw data can be post-processed and analyzed automatically, thereby eliminating observer subjectivity, and providing quantitative results with which worker populations may be evaluated. The following components were incorporated into the system design: a National Instruments® AI-16XE-50 DAQ Card; a National Instruments® PCI-1411 framegrabber, a National Instruments® SC-2043-SG, strain gauge accessory; a Miniature CMOS Camera (⅓ inch format, 510×492 array size); a load cell (Interface™); and a photoplethysmograph transducer, power supply, and amplifier (Biopac).

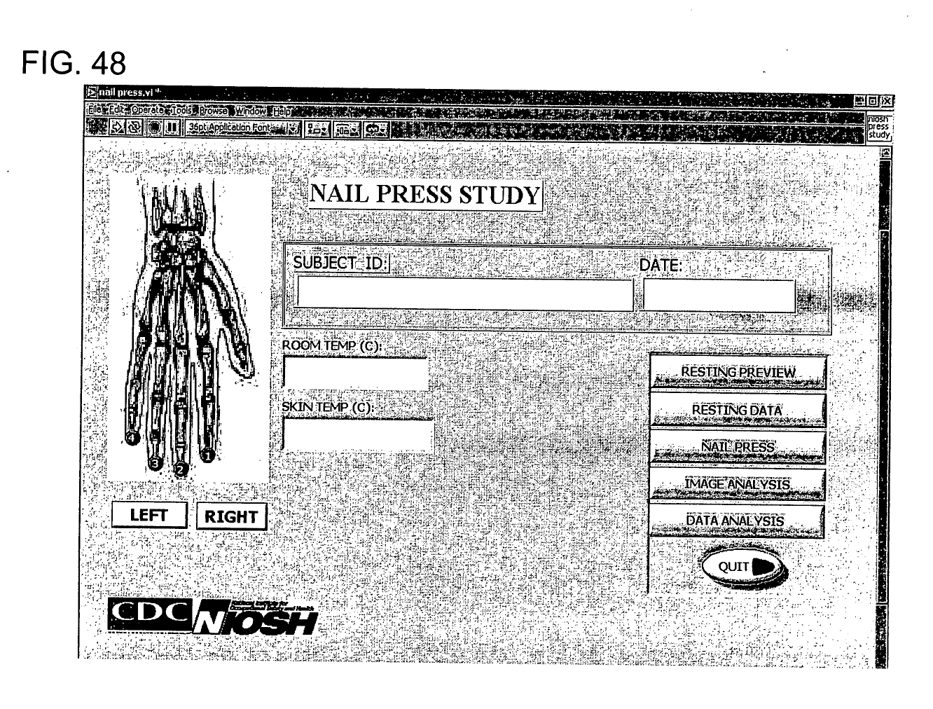

An interactive front panel (as shown in FIG. 48) can provide access to a variety of software methods through respective visual interfaces (VI's) within a waiting state machine. Relevant subject data is entered by the operator, including ID number, finger and room temperature, handedness and finger designation, which are incorporated into the appropriate file header or directory designation. A variety of VIs may then be accessed including those that follow.

A RESTING PREVIEW VI provides image and transducer verification, and allows focus and finger placement adjustments to be made while familiarizing the subject with the procedure.

A RESTING DATA VI collects 30-60 seconds of analog input data, providing baseline measurements of the pulse volume and resting finger force. From these measurements, the resting mean global variable is populated, and target force values for subsequent use are computed from this value. Data is gathered at a high collection rate (1024 Hz) and provides an additional resource of data for any subsequently desired frequency analysis measurements requiring higher resolution.

A NAIL PRESS VI utilizes its front panel interface to instruct and prompt the subject accordingly. Data collections consists of two sequences but starts only after the occurrence of a hardware trigger. This trigger starts a counter on the DAQ card, which generates a square pulse wave (1 Hz) that serves to initiate each buffer of the mage acquisition. During the initial sequence (the first 10s), the interface provides an audible prompt and visual instructions to “Rest and Relax” while resting images and analog input data (128 Hz) are gathered. The subject is then instructed to “Press and Hold” (i.e., press the finger downwardly against lever 132) in order to reach a desired target force, calculated by adding a load cell calibrated force to the resting mean global value. Once the target is reached, a countdown of 10 seconds begins, during which the subject maintains his/her target force level. Images and analog input data (128 Hz) are gathered during the entire “Press and Hold” sequence. The subject is then prompted to “Rest and Relax” (i.e. relax pressure in the finger) for 30-60 additional seconds of data collection. A browser (for example, including the screen shots in FIGS. 12 and 36) is created which displays the images acquired during the press for verification of analysis results. All images and raw data are written to file for post-processing and analysis. The data is time-stamped. for timing verification purposes. The use of software synchronization was not problematic at the low image acquisition rate (1 Hz); however, the use of hardware synchronization, which provides resolution in the order of nanoseconds, can be used at higher collection rates. The accuracy of the results can be improved by increasing the resolution of the image acquisition (i.e. increasing the image acquisition rate by using a faster camera).

Characteristic force and blood volume responses during the press can be displayed as shown in FIG. 38. The finger press sequence may be reviewed within the IMAGE ANALYSIS VI, which cycles through the acquired images at a user-defined speed. As each image is displayed, a histogram is generated and data from this report is unbundled and saved for further analysis. An example of images and their respective histograms are shown in FIGS. 13A and 13B. Although acquired as RGB images, the histograms can be generated from their 8 bit representations. From the histogram data, selections are made for the spectrum groups demonstrating the most significant change during the press (for example, the spectrum groups having the greatest point to point slopes as shown in FIG. 15), and an evaluation of the pixel numbers within these groups over time generates a blood flow curve (for example, the blood flow signals shown in FIG. 16) which, not surprisingly, resembles the curves of the force and the photoplethysmograph blood volume results.

An algorithm was developed and incorporated into both the IMAGE ANALYSIS VI and the DATA ANALYSIS VI to analyze the collected data. The typical image, force, and blood volume data all revealed steep slopes during the finger press; therefore, the data were analyzed by taking step derivatives along the course of each press. Plotting the absolute values of these results reveals the areas of maximal change as defined by sharp peaks above baseline. The times corresponding to these peaks were calculated, and represent the times of maximal change for each press. Results following the application of the step differentiation automated method to image blood flow data, and the force and photoplethysmograph blood volume data respectively are shown in FIGS. 37A and 37B.

### EXAMPLE 47

### Details of Additional Exemplary Experimental Results of Evaluation of Physiological Conditions from Analysis of an Induced Change in Blood Flow of a Feature

In this example, apparatus 500 shown in FIG. 5 was utilized to collect blood flow data from multiple subjects for the purpose of evaluating physiological conditions of the subjects. Software using the described methods was used to record and analyze force and blood volume data in which the source of the force application was a linear stepper motor. A blood flow evaluation was conducted on the left and right hand fingers of 43 subjects ranging in age from 31-59. Blood flow characteristics were determined using the described methods. Some of the results of the evaluation are shown in Tables 4.

TABLE 4
Changes in Blood Flow Characteristics

| Blood Flow Characteristics | Change | Significance |
|---|---|---|
| DC pre R vs. DC post R | Increase | p < 0.001 |
| AC pre R vs. AC post R | Increase | p < 0.001 |
| DC pre L vs. DC post L | Increase | p < 0.001 |
| AC pre L vs. AC post L | Increase | p < 0.001 |
| NPV pre R vs. NPV post R | Decrease | p < 0.001 |
| DNPV pre R vs. DNPV post R | Decrease | p < 0.001 |
| NPV pre L vs. NPV post L | Decrease | p = 0.004 |
| DNPV pre L vs. DNPV post L | Decrease | p < 0.001 |

R = right hand,
L = Left Hand
DC = direct current component of PPG signal
AC = Alternating current component of PPG signal
NPV = normalized pulse,
DNPV = double normalized pulse

As shown in the Table 4, the normalized pulse volume and double normalized pulse volume significantly decreased following the press (application of force). Additionally, there was a significant difference between the left and right hand return time (p=0.026), with the return time being longer for the left hand. Similarly, there was a correlation of age with force shoulder of the left hand following the finger press, with the shoulder being greater in subjects younger than 45 versus subjects 45 or older. This can reflect decreasing tissue compliance with age as shown by the overall return time of the left hand being significantly (p=0.029) longer in subjects less than 45. Finger volume measurements were also performed and the finger volume of the right hand is significantly greater than the left hand (p<0.001), reflecting increased blood flow to the right hand.

### EXAMPLE 48

### Exemplary Computer System for Conducting Analysis

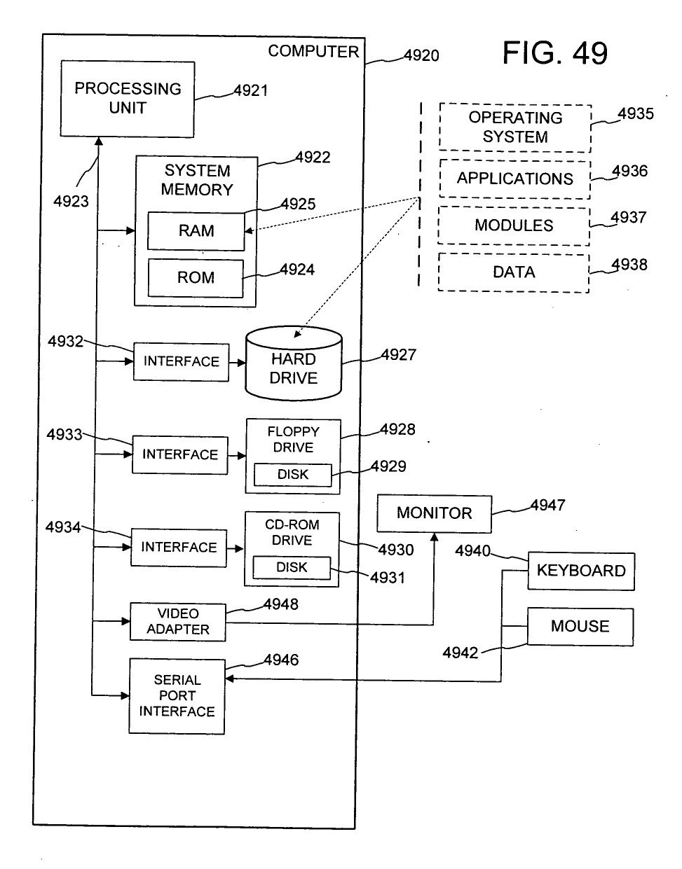

FIG. 49 and the following discussion provide a brief, general description of a suitable computing environment for the software (for example, computer programs) described above. The methods described above can be implemented in computer-executable instructions organized in program modules. The program modules can include the routines, programs, objects, components, and data structures that perform the tasks and implement the data types for implementing the techniques described above.

While FIG. 49 shows a typical configuration of a desktop computer, the technologies may be implemented in other computer system configurations, including multiprocessor systems, microprocessor-based or programmable consumer electronics, minicomputers, mainframe computers, and the like. The technologies may also be used in distributed computing environments where tasks are performed in parallel by processing devices to enhance performance. For example, tasks related to measuring characteristics of candidate anomalies can be performed simultaneously on multiple computers, multiple processors in a single computer, or both. In a distributed computing environment, program modules may be located in both local and remote memory storage devices.

The computer system shown in FIG. 49 is suitable for implementing the technologies described herein and includes a computer 4920, with a processing unit 4921, a system memory 4922, and a system bus 4923 that interconnects various system components, including the system memory to the processing unit 4921. The system bus may comprise any of several types of bus structures including a memory bus or memory controller, a peripheral bus, and a local bus using a bus architecture. The system memory includes read only memory (ROM) 4924 and random access memory (RAM) 4925. A nonvolatile system (for example, BIOS) can be stored in ROM 4924 and contains the basic routines for transferring information between elements within the personal computer 4920, such as during start-up. The personal computer 4920 can further include a hard disk drive 4927, a magnetic disk drive 4928, for example, to read from or write to a removable disk 4929, and an optical disk drive 4930, for example, for reading a CD-ROM disk 4931 or to read from or write to other optical media. The hard disk drive 4927, magnetic disk drive 4928, and optical disk 4930 are connected to the system bus 4923 by a hard disk drive interface 4932, a magnetic disk drive interface 4933, and an optical drive interface 4934, respectively. The drives and their associated computer-readable media provide nonvolatile storage of data, data structures, computer-executable instructions (including program code such as dynamic link libraries and executable files), and the like for the personal computer 4920. Although the description of computer-readable media above refers to a hard disk, a removable magnetic disk, and a CD, it can also include other types of media that are readable by a computer, such as magnetic cassettes, flash memory cards, digital video disks, and the like.

A number of program modules may be stored in the drives and RAM 4925, including an operating system 4935, one or more application programs 4936, other program modules 4937, and program data 4938. A user may enter commands and information into the personal computer 4920 through a keyboard 4940 and pointing device, such as a mouse 4942. Other input devices (not shown) may include a microphone, joystick, game pad, satellite dish, scanner, or the like. These and other input devices are often connected to the processing unit 4921 through a serial port interface 4946 that is coupled to the system bus, but may be connected by other interfaces, such as a parallel port, game port, or a universal serial bus (USB). A monitor 4947 or other type of display device is also connected to the system bus 4923 via an interface, such as a display controller or video adapter 4948. In addition to the monitor, personal computers typically include other peripheral output devices (not shown), such as speakers and printers. Furthermore, pci slots (not shown) can be used for integration of the above computer system with any of the apparatuses described (for example, the framegrabber and data acquisition card of a blood flow apparatus can be connected into the pci slots of the computer system).

The above computer system is provided merely as an example. The technologies can be implemented in a wide variety of other configurations, including a microcontroller interface for decreased size and cost, and increased portability. Further, a wide variety of approaches for collecting and analyzing data related to processing blood flow is possible. For example, the data can be collected, blood flow characteristics determined and classified, and the results presented on different computer systems as appropriate. In addition, various software aspects can be implemented in hardware, and vice versa.

### EXAMPLE 49

### Exemplary Methods

Any of the methods described herein can be performed by software executed by software in an automated system (for example, a computer system). Fully-automatic (for example, without human intervention) or semi-automatic operation (for example, computer processing assisted by human intervention) can be supported. User intervention may be desired in some cases, such as to adjust parameters or consider results.

Such software can be stored on one or more computer-readable media comprising computer-executable instructions for performing the described actions.

### EXAMPLE 50

### Exemplary Embodiments

### Embodiment A

One or more computer readable media comprising computer-readable instructions for performing:
receiving a plurality of digital representations of blood flow within a feature over time;
analyzing the digital representations to determine a signal representative of the blood flow.

### Embodiment B

The one or more computer readable media of embodiment A, wherein analyzing the digital representations to determine a signal representative of blood flow comprises:
classifying digital representation components of the digital representations as being of respective groups of components; and
analyzing the respective groups of components in the digital representations to determine the signal representative of blood flow.

### Embodiment C

The one or more computer-readable media of embodiment A, wherein receiving digital representations of the blood flow comprises receiving digital representations of the blood flow underneath the nail of a digit.

### Embodiment D

The one or more computer-readable media of embodiment A, wherein the digital representations of blood flow are received from a camera.

### Embodiment E

The one or more computer-readable media of Embodiment A, wherein the digital representations of blood flow are RGB color digital representations.

### Embodiment F

The one or more computer-readable media of embodiment E, wherein the RGB color digital representations are converted to grayscale.

### Embodiment G

The one or more computer-readable media of embodiment B, wherein the classifying digital representation components comprises assigning components of a digital representation into groups of components based on at least one criteria selected from the group consisting of brightness and spectrum.

### Embodiment H

The one or more computer-readable media of embodiment B, wherein the digital representation components comprise pixels and the classifying digital representation components comprises classifying the pixels as being of respective spectrum component groups.

### Embodiment I

The one or more computer-readable media of embodiment B, wherein analyzing the respective groups of components to determine the signal representative of blood flow comprises:
detecting one or more changes in the number of components in one or more groups of components in the digital representations; and
generating a signal representative of blood flow based on the changes in the number of components.

### Embodiment J

The one or more computer-readable media of embodiment A, further comprising computer-executable instructions for performing:
presenting a visual depiction of at least one digital representation of the blood flow.

### Embodiment K

The one or more computer-readable media of embodiment B further comprising computer-executable instructions for performing:
presenting a visual depiction of the classified components of at least one digital representation.

### Embodiment L

The one or more computer-readable media of embodiment I, further comprising computer-executable instructions for performing:
presenting a visual depiction of the detected one or more changes in the number of components in one or more groups of components in the digital representations.

### Embodiment M

The one or more computer-readable media of claim Embodiment A, further comprising computer-executable instructions for performing:
presenting a visual depiction of the signal.

### Embodiment N

The one or more computer-readable media of embodiment I, further comprising computer-executable instructions for performing:
in a software user interface, presenting visual depictions comprising at least one depiction selected from the group consisting of:
   a visual depiction of at least one digital representation of the blood flow;
   a visual depiction of the classified components of at least one digital representation;
   a visual depiction of the detected one or more changes in the number of components in one or more groups of components in the digital representations; and
   a visual depiction of the signal.

### Embodiment M

A method for evaluating blood flow within a feature of a subject, the method comprising:
applying a force to the feature so as to cause a change in the blood flow of the digit;
measuring blood flow within the feature;
determining at least one blood flow characteristic from the measured blood flow corresponding to the change in blood flow; and
analyzing the at least one characteristic to evaluate a physiological condition of the subject.

### Embodiment N

A method according to embodiment M, further comprising reducing the applied force to cause a change in blood flow within the feature.

### Embodiment O

A method according to embodiment M, wherein the force is applied to the feature by a force-applying mechanism.

### Embodiment P

A method according to embodiment M, wherein the force is applied to the feature by the subject pressing the pad of said digit against a surface.

### Embodiment Q

A method according to embodiment N, wherein determining at least one blood flow characteristic based on the change in blood flow comprises determining a rate of return of blood flow into the feature.

### Embodiment R

A method according to embodiment N, wherein determining at least one blood flow characteristic based on the change in blood flow comprises determining a difference between the amount of blood flow before the force is applied and the amount of blood flow after the force is reduced.

### Embodiment S

A method according to embodiment N, wherein blood flow is measured by a photoplethysmograph detector.

### Embodiment T

A method according to embodiment S, wherein the measured blood flow is represented by a photoplethysmograph signal.

### Embodiment U

A method according to embodiment T, wherein the at least one blood flow characteristic comprises a characteristic of the photoplethysmograph signal of the measured blood flow.

### Embodiment V

A method according to embodiment U, wherein determining at least one blood flow characteristic based on the change in blood flow comprises determining a difference between at least one characteristic of the photoplethysmograph signal before the force is applied and the at least one characteristic after the force is reduced.

### Embodiment W

A method according to embodiment V, wherein the at least one characteristic of the photoplethysmograph signal comprises at least one parameter from the group consisting of:
direct current component;
alternating current component;
normalized pulse volume; and
double normalized pulse volume.

### Embodiment X

A method according to embodiment W, wherein the normalized pulse volume comprises the pulsatile component of the photoplethysmograph signal divided by the baseline transmitted light.

### Embodiment Y

A method according to embodiment X, wherein the double normalized pulse volume comprises the pulsatile component of total blood volume divided by the total amount of blood in both arterial and venous blood vessels.

### Embodiment Z

A method according to embodiment N, further comprising measuring the force.

### Embodiment AA

A method according to embodiment Z, wherein the force is measured by a load cell.

### Embodiment BB

A method according to embodiment Z, wherein the measured force is represented by a force signal.

### Embodiment CC

A method according to embodiment BB, wherein determining at least one blood flow characteristic based on the change in blood flow comprises determining a time interval representative of blood volume return.

### Embodiment DD

A method according to embodiment CC, wherein the time interval representative of blood volume return comprises the interval between a time point of the measured force signal and a time point at which the measured blood flow has returned to at least levels before the force is applied.

### Embodiment EE

A method according to embodiment DD, wherein the time point from the measured force signal comprises the time point at force release.

### Embodiment FF

A method according to embodiment DD, wherein the time point from the measured force signal comprises the time point at which the rate of change of the reduced force is greatest.

### Embodiment GG

A method according to embodiment CC, wherein the time interval representative of blood volume return comprises the interval between and a time point of the measured force signal and a time point at which the rate of change of the blood flow after the force has been released is greatest.

### Embodiment HH

A method according to embodiment GG, wherein the time point from the measured force signal comprises the time point at force release.

### Embodiment II

A method according to embodiment GG, wherein the time point from the measured force signal comprises the time point at which the rate of change of the reduced force is greatest.

### Embodiment JJ

A method according to embodiment M, wherein measuring blood flow comprises:
acquiring digital representations of the feature; and
generating a signal that is representative of blood flow within the feature.

### Embodiment KK

A method according to embodiment M, wherein analyzing the at least one characteristic to evaluate a physiological condition of the subject comprises comparing the at least one blood flow characteristic of the subject with at least one blood flow characteristic of at least one subject having at least one specified physiological condition.

### Embodiment LL

A method according to embodiment M, further comprising presenting a visual depiction of the at least one blood flow characteristic.

### Embodiment MM

A method according to embodiment KK, further comprising presenting a visual depiction of the at least one blood flow characteristic of the subject and the at least one blood flow characteristic of the at least one subject having the at least one specified physiological condition.

### Embodiment NN

A method according to embodiment M, further comprising presenting a visual depiction of the measured blood flow.

### Embodiment OO

A method according to embodiment embodiment Z, further comprising presenting a visual depiction of the measured force.

### Embodiment PP

A method according to embodiment M, further comprising determining a target force sufficient to prevent blood flow to the digit.

### Embodiment QQ

A method according to embodiment PP, wherein applying a force to the digit comprises applying the target force.

### Embodiment RR

A method according to embodiment M, wherein the physiological condition is handedness.

### Embodiment SS

A method according to embodiment M, wherein the physiological condition is age.

### Embodiment TT

A method according to embodiment M, wherein the physiological condition is a condition demonstrating critical changes in peripheral circulation.

### Embodiment UU

A method according to embodiment TT, wherein the physiological condition is hand arm vibration syndrome (HAVS).

### Embodiment VV

A method according to embodiment TT, wherein the physiological condition is peripheral vascular disease.

### Embodiment WW

One or more computer-readable media comprising computer-executable instructions for performing the method of embodiment M.

### Embodiment XX

One or more computer-readable media comprising computer-executable instructions for performing:
receiving a photoplethysmograph signal from a subject;
determining the stability of the signal; and
using the signal for analysis in evaluating a physiological condition of a subject if the stability of the signal is acceptable.

### Embodiment YY

The one or more computer-readable media of embodiment XX wherein a photoplethysmograph signal is received by a detector unit for detecting PPG signals.

### Embodiment ZZ

The one or more computer-readable media of embodiment XX further comprising removing distortion from the signal.

### Embodiment AAA

The one or more computer-readable media of embodiment ZZ wherein removing distortion comprises applying a hanning window.

### Embodiment BBB

The one or more computer-readable media of embodiment XX wherein the stability of the signal is determined by at least the slope of the direct current component of the signal.

### Embodiment CCC

The one or more computer-readable media of embodiment ZZ wherein the stability of the signal is determined by at least analyzing peaks and valleys in the alternating current component of the signal.

### Embodiment DDD

The one or more computer-readable media of embodiment CCC wherein analyzing peaks and valleys in the alternating current component of the signal comprises determining at least one pulse parameter selected from the group consisting of:
a stable pulse width;
a stable pulse area; and
a stable pulse height.

### Embodiment EEE

The one or more computer-readable media of embodiment DDD wherein determining the stable pulse width comprises analyzing valley-to-valley times by at least one statistical measurement.

### Embodiment FFF

The one or more computer-readable media of embodiment EEE wherein the at least one statistical measurement comprises at least one statistical measurement selected from the group consisting of:
mean;
correlation;
variance; and
standard deviation.

### Embodiment GGG

The one or more computer-readable media of embodiment EEE wherein determining the stable pulse area comprises analyzing valley-to-valley integrals by at least one statistical measurement.

### Embodiment HHH

The one or more computer-readable media of embodiment GGG wherein the at least one statistical measurement comprises at least one statistical measurement selected from the group consisting of:
mean;
correlation;
variance; and
standard deviation.

### Embodiment III

The one or more computer-readable media of embodiment DDD wherein determining the stable pulse height comprises analyzing valley-to-peak distances by at least one statistical measurement.

### Embodiment JJJ

The one or more computer-readable media of embodiment III wherein the at least one statistical measurement comprises at least one statistical measurement selected from the group consisting of:
mean;
correlation;
variance; and
standard deviation.

### Embodiment KKK

One or more computer-readable media comprising computer executable instructions for performing:
receiving a photoplethysmograph signal from a resting subject;
determining a mean pulse of the signal based on linear associations between pulses within the signal; and
based on at least the mean pulse of the signal, evaluating a physiological condition of a subject.

### Embodiment LLL

The one or more computer-readable media of embodiment KKK wherein the photoplethysmograph signal from a resting subject is stable.

### Embodiment MMM

The one or more computer-readable media of embodiment KKK wherein the linear associations are measured by correlation coefficients.

### Embodiment NNN

The one or more computer-readable media of embodiment MMM wherein determining a mean pulse of the signal comprises:
determining a plurality of correlation coefficients for each pulse in the signal with other pulses in the signal;
categorizing each pulse as being associated with pulses in the signal based at least on the plurality of correlation coefficients; and
determining a mean pulse based at least on the associated pulses.

### Embodiment OOO

The one or more computer-readable media of embodiment KKK further comprising normalizing the mean pulse.

### Embodiment PPP

The one or more computer-readable media of embodiment KKK wherein evaluating a physiological condition of a subject comprises:
determining at least one pulse parameter of the mean pulse of the signal; and
determining at least one physiological condition of a subject based on the at least one pulse parameter.

### Embodiment QQQ

The one or more computer-readable media of embodiment PPP wherein determining at least one pulse parameter of the mean pulse of the signal comprises determining at least one parameter selected from the group consisting of:
minimum rise time;
stiffness index;
frequency analysis of harmonics; and
normalized pulse shape analysis.

### Embodiment RRR

The one or more computer-readable media of embodiment KKK, wherein the physiological condition is age.

### Embodiment SSS

The one or more computer-readable media of embodiment KKK, wherein the physiological condition is a condition demonstrating critical changes in peripheral circulation.

### Embodiment TTT

The one or more computer-readable media of embodiment SSS, wherein the physiological condition is peripheral vascular disease.

### Embodiment UUU

A system for evaluating a physiological condition of a subject, the system comprising:
means for applying a force to a digit of the subject so as to cause a change in the blood flow of the digit;
means for measuring blood flow within the digit;
means for calculating one or more characteristics of the measured blood flow corresponding to the change in blood flow;
means for evaluating a physiological condition of a subject based at least on the one or more characteristics.

### Embodiment VVV

The system of embodiment UUU, further comprising means for reducing the applied force to cause a change in blood flow within the digit.

### Embodiment WWW

The system of embodiment UUU, further comprising means for measuring the force.

### Alternatives

Having illustrated and described the principles of the invention in exemplary embodiments, it should be apparent to those skilled in the art that the described examples are illustrative embodiments and can be modified in arrangement and detail without departing from such principles.

Although some of the examples describe peripheral blood flow and detecting blood flow characteristics from a digit, the technologies can be applied to other anatomical structures as well. For example implementations can be applied to any peripheral anatomical structure such as a hand, arm, foot, leg, head, ear, nose or any other peripheral anatomical structure found in human beings or other vertebrates. Anatomical structures can also include any other anatomical structure or portion thereof found in human beings or other vertebrates in which blood flows and is not necessarily limited to peripheral blood flow analysis.

In view of the many possible embodiments to which the principles of the invention may be applied, it should be understood that the illustrative embodiments are intended to teach these principles and are not intended to be a limitation on the scope of the invention. We therefore claim as our invention all that comes within the scope and spirit of the following claims and their equivalents.

We claim:

1. An apparatus for evaluating blood flow within a digit of a subject, said apparatus comprising:
an image-capturing device that is operable to acquire digital representations of the blood flow within said digit; and
a processor configured to receive digital representations from the image-capturing device and generate a signal that is representative of blood flow, wherein the signal can be used to determine a physiological condition of a subject.

2. An apparatus according to claim 1, wherein the image-capturing device comprises a camera.

3. An apparatus according to claim 1, further comprising a force applicator that is adapted to apply a force to said digit.

4. An apparatus according to claim 3, wherein the force applicator comprises a linear stepper motor that increases and decreases the force applied to the digit.

5. An apparatus according to claim 4, wherein the force applicator further comprises a transparent pressing surface; and
wherein the linear stepper motor is coupled to the pressing surface and operable to cause the pressing surface to press against the digit and move away from the digit so as to increase and decrease the force applied to the digit; and
wherein the image-capturing device is positioned to capture images of the blood flow through the transparent pressing surface

6. An apparatus according to claim 1, further comprising a digit-engagable surface that is adapted to receive a digit, wherein the user presses against the digit-engagable surface to apply a force to said digit.

7. An apparatus according to claim 2, further comprising a focus wheel coupled to the camera.

8. An apparatus according to claim 1, further comprising a photoplethysmograph detector for detecting a photoplethysmograph signal of the digit.

9. An apparatus according to claim 8, further comprising a digit engagable surface that is adapted to receive a digit, and wherein the photoplethysmograph detector is located on the opposite side of the surface from the finger, and the digit-engagable surface is transparent to light detected by the photoplethysmograph detector.

10. One or more computer readable media comprising computer-readable instructions for performing:
receiving a plurality of digital representations of blood flow within a feature over time;
analyzing the digital representations to determine a signal representative of the blood flow.

11-23. (canceled)

24. An apparatus for evaluating blood flow within a digit of a subject, said apparatus comprising:
a force measuring device for measuring force applied to the digit;
a blood flow detector for generating a blood flow signal representative of blood flow in the digit; and
a signal processor configured to:
receive signals from the force measuring device and the blood flow detector;
generate at least one blood flow characteristic based on at least one of:
the signal from the blood flow detector and the signal from the force measuring device;
the signal from the blood flow detector; or
the signal from the force measuring device;
evaluate at least one physiological condition of the subject based on the at least one blood flow characteristic.

25. An apparatus according to claim 24, wherein the force measuring device is a load cell.

26. An apparatus according to claim 24, wherein the blood flow detector is a photoplethysmograph detector.

27. An apparatus according to claim 26, wherein the photoplethysmograph detector is a photoplethysmograph transducer.

28. An apparatus according to claim 24, wherein the blood flow detector comprises:
an image-capturing device operable to acquire digital representations of the blood flow in the digit; and
a digital representation processor configured to receive digital representations from the image-capturing device and generate said blood flow signal from said digital representations, wherein the signal processor receives said blood flow signal from the digital representation processor.

29. An apparatus according to claim 24, further comprising a digit-engagable surface adapted to receive the digit, and wherein the force measuring device measures the force applied by the digit to the digit-engagable surface.

30. An apparatus according to claim 29, wherein the blood flow detector is a photoplethysmograph detector that is located below the digit-engagable surface, and wherein the digit-engagable surface is transparent to light detected by the photoplethysmograph detector.

31. An apparatus according to claim 29, further comprising a pivotable lever, and wherein said digit-engagable surface is a surface of the pivotable lever.

32. An apparatus according to claim 29, wherein the force measuring device is a load cell located to receive the force applied to the digit-engagable surface.

33. An apparatus according to claim 29, further comprising:
a movable pressing surface; and
a motor coupled to the pressing surface and operable to cause the pressing surface to press the digit against the digit-engagable surface so as to apply the force and to move away from the digit so as to reduce the force.

34. An apparatus according to claim 33, further comprising:
a safety mechanism operable to prevent the application of excessive force to the digit if the force applied by the pressing surface exceeds a predetermined threshold.

35. An apparatus according to claim 33, wherein the blood flow detector comprises:
an image-capturing device that is operable to acquire digital representations of the blood flow within said digit; and
a digital representation processor configured to receive digital representations from the image-capturing device and generate said blood flow signal, wherein the signal processor receives said blood flow signal from the digital representation processor; and
wherein the pressing surface is transparent to allow the image-capturing device to capture images of the blood flow through the pressing surface.

36. An apparatus according to claim 24, wherein the at least one characteristic comprises at least one characteristic selected from the group consisting of:
a rate of return of blood flow within the digit;
a difference between blood flow before the force is applied and blood flow after reducing the force; and
a time interval representative of blood volume return.

37. An apparatus according to claim 24, wherein the signal processor is configured to determine at least one physiological condition based on the at least one blood flow characteristic.

38. An apparatus according to claim 37, wherein the physiological condition is handedness.

39. An apparatus according to claim 37, wherein the physiological condition is age.

40. An apparatus according to claim 37, wherein the physiological condition is a condition demonstrating critical changes in peripheral circulation.

41. An apparatus according to claim 40, wherein the physiological condition is hand arm vibration syndrome (HAVS).

42. An apparatus according to claim 40, wherein the physiological condition is peripheral vascular disease.

43. A method for evaluating blood flow within a feature of a subject, the method comprising:
applying a force to the feature so as to cause a change in the blood flow of the digit;
measuring blood flow within the feature;
determining at least one blood flow characteristic from the measured blood flow corresponding to the change in blood flow; and
analyzing the at least one characteristic to evaluate a physiological condition of the subject.

44-79. (canceled)

80. One or more computer-readable media comprising computer-executable instructions for performing:
receiving a photoplethysmograph signal from a subject;
determining the stability of the signal; and
using the signal for analysis in evaluating a physiological condition of a subject if the stability of the signal is acceptable.

81-92. (canceled)

93. One or more computer-readable media comprising computer executable instructions for performing:
receiving a photoplethysmograph signal from a resting subject;
determining a mean pulse of the signal based on linear associations between pulses within the signal; and
based on at least the mean pulse of the signal, evaluating a physiological condition of a subject.

94-102. (canceled)

103. A system for evaluating a physiological condition of a subject, the system comprising:
means for applying a force to a digit of the subject so as to cause a change in the blood flow of the digit;
means for measuring blood flow within the digit;
means for calculating one or more characteristics of the measured blood flow corresponding to the change in blood flow;
means for evaluating a physiological condition of a subject based at least on the one or more characteristics.

104-105. (canceled)
